(19) United States
(12) Patent Application Publication
(10) Pub. No.: US 2019/0046486 A1
(43) Pub. Date: Feb. 14, 2019

(54) COMPOSITIONS AND METHODS FOR THE TREATMENT OF NEURONAL INJURY

(71) Applicant: AXCELLA HEALTH INC., Cambridge, MA (US)

(72) Inventors: Gianluca De Rienzo, Brookline, MA (US); Raffi Afeyan, Boston, MA (US); Sean Carroll, Cambridge, MA (US); Matthew Russell, West Newton, MA (US)

(21) Appl. No.: 16/103,200

(22) Filed: Aug. 14, 2018

Related U.S. Application Data
(60) Provisional application No. 62/697,690, filed on Jul. 13, 2018.
(60) Provisional application No. 62/614,198, filed on Jan. 5, 2018.
(60) Provisional application No. 62/545,364, filed on Aug. 14, 2017.

(51) Int. Cl. A61K 31/198; A61K 38/05; A61K 38/06; A61P 25/28; A23L 33/175; A23L 33/18

(52) U.S. Cl. CPC A61K 31/198; A61K 38/05; A61K 38/06; A61P 25/28; A23L 33/175; A23L 33/18; A23V 2250/306; A23V 2200/322; A23V 2250/0634; A23V 2250/0628; A23V 2250/0626; A23V 2250/0654; A23V 2002/00

(57) ABSTRACT

This disclosure provides compositions and methods for treating or preventing neuronal injury in a subject, e.g., a subject at risk of or having traumatic brain injury or stroke.

### RELATED APPLICATIONS

This application claims priority to U.S. Ser. No. 62/545,364 filed Aug. 14, 2017, U.S. Ser. No. 62/614,198 filed Jan. 5, 2018, and U.S. Ser. No. 62/697,690 filed Jul. 13, 2018, the contents of which are each incorporated herein by reference in their entireties.

### BACKGROUND

Traumatic brain injury (TBI) is a leading cause of death and disability among children and young adults in the United States. An estimated 3 million Americans sustain a TBI annually. Traumatic brain injury (TBI) is characterized by a disruption in the normal function of the brain due to an acute external force. Although TBI can be mild and not cause long-term disability, TBI is also a major cause of disability and even death worldwide. Despite the incidence of TBI, current treatments focus on stabilizing the patient without preventing further injury and do not address the neuronal injury caused by trauma.

Nearly 800,000 people in the United States have a stroke every year, with about three in four being first-time strokes. Stroke is the fifth leading cause of death in the United States, killing nearly 130,000 people a year, and is a leading cause of long-term disability. An ischemic stroke occurs when a clot or a mass blocks a blood vessel, cutting off blood flow to a part of the brain. Approximately 87% of strokes are classified as ischemic. Current therapies for stroke include intravenous thrombolytics, which have a limited window of efficacy after stroke occurrence, side effects (e.g., risk of intracerebral hemorrhage), and contra-indications (e.g., anti-coagulant use or uncontrolled hypertension).

Thus, there is still a need for agents for treating neuronal injury, such as TBI and stroke.

### SUMMARY

Provided herein is a composition (an Active Moiety) including amino acid entities that is useful for improving one or both of neuronal function or cognitive function, e.g., a subject having neuronal injury. The composition can be used in a method of treating (e.g., reversing, reducing, ameliorating, or preventing) neuronal injury (e.g., traumatic brain injury (TBI) or stroke) in a subject (e.g, a human) in need thereof. The method can further include monitoring the subject for an improvement in one or more symptoms of neuronal injury after administration of the composition, e.g., such that the subject exhibits an improvement in the one or more symptoms after administration of the composition.

In one aspect, the invention features a composition (Active Moiety) comprising, consisting of, or consisting essentially of: a) a Branched Chain Amino Acid (BCAA) entity chosen from a leucine amino acid entity, an isoleucine amino acid entity, a valine amino acid entity, or a combination of two or three BCAA entities; b) a N-acetylcysteine (NAC) entity; and c) an acetyl-1-carnitine (ALCAR) entity; in which at least one amino acid entity of (a)-(c) is not provided as a peptide of more than 20 amino acid residues in length.

In some embodiments, glutamine is absent, or if present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (in dry form).

In some embodiments, the composition further comprises (d) a creatine entity. In some embodiments, the creatine entity is not provided as a peptide of more than 20 amino acid residues in length. In some embodiments, the creatine entity is selected from Table 1. In some embodiments, the creatine entity is creatine or a salt thereof or a dipeptide or salt thereof, or a tripeptide or salt thereof, comprising creatine.

In another aspect, the invention features a composition comprising, consisting of, or consisting essentially of: a) a Branched Chain Amino Acid (BCAA) entity chosen from a leucine amino acid entity, an isoleucine amino acid entity, a valine amino acid entity, or a combination of two or three BCAA entities; b) a N-acetylcysteine (NAC) entity; and c) acetyl-1-carnitine (ALCAR) or a salt thereof or a dipeptide or salt thereof, or a tripeptide or salt thereof, comprising ALCAR; in which at least one amino acid entity of (a)-(c) is not provided as a peptide of more than 20 amino acid residues in length.

In another aspect, the invention features a composition comprising, consisting of, or consisting essentially of: a) leucine, isoleucine, and valine; b) N-acetylcysteine (NAC); and c) acetyl-L-carnitine and/or creatine.

In some embodiments, two, three, four, or five amino acid entities of (a)-(c) is not provided as a peptide of more than 20 amino acid residues in length.

In some embodiments, the total wt. % of (a)-(c) (e.g., three, four, or five amino acid entities in (a)-(c)) is greater than the total wt. % of other protein components (e.g., whey protein) or non-protein components (or both) in the composition (e.g., in dry form), e.g., the total wt. % (a)-(c) is at least: 50 wt. %, 75 wt. %, or 90 wt. % of the total wt. of protein components or total components in the composition (e.g., in dry form).

In some embodiments, one, two, three, four, or five amino acid entities in (a)-(c) are in one or both of free amino acid form or salt amino acid form in the composition, e.g., at least: 35 wt. %, 40 wt. %, 42 wt. %, 45 wt. %, 50 wt. %, 75 wt. %, 80 wt. %, 90 wt. %, or more, of the total wt. of the composition (e.g., in dry form) is three, four, or five amino acid entities in (a)-(c) in one or both of free amino acid form or salt amino acid form in the composition.

In some embodiments, the composition does not comprise a peptide of more than 20 amino acid residues in length (e.g., whey protein), or if a peptide of more than 20 amino acid residues in length is present, the peptide is present at less than: 10 wt. %, 5 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form).

In some embodiments, the composition comprises a combination of 19 or fewer, 18 or fewer, 15 or fewer, 12 or fewer, or 10 or fewer amino acid entities. In some embodiments, the combination comprises at least: 42 wt. %, 75 wt. %, or 90 wt. % of the total wt. of protein components or total components in the composition (e.g., in dry form).

In some embodiments, the wt. % of the BCAA entity or combination of two or three BCAA entities (e.g., one, two, or three of a leucine amino acid entity, an isoleucine amino acid entity, or a valine amino acid entity), the NAC entity, and the ALCAR entity (e.g., the ALCAR or a salt thereof or a dipeptide or salt thereof, or a tripeptide or salt thereof, comprising ALCAR) in combination is at least 20 wt. % of the total wt. of protein components or total components in the composition (in dry form), e.g., the wt. % of the BCAA entity or combination of two or three BCAA entities, the NAC entity, and the ALCAR entity in combination is at least: 30 wt. %, 40 wt. %, 50 wt. %, 60 wt. %, 70 wt. %, 80 wt. %, 90 wt. %, or more of total wt. of the protein components or total components in the composition (in dry form).

In some embodiments, the wt. % of the NAC entity is at least 3 wt. % of the total wt. of the protein components or total components in the composition (in dry form), e.g., the wt. % of the NAC entity is at least: 4 wt. %, 5 wt. %, 6 wt. %, 7 wt. %, or 8 wt. % of the total wt. of protein components or total components in the composition. Optionally, NAC is not more than 25 wt. % of the total wt. of protein components or total components in the composition in dry form.

In some embodiments, the wt. % of the ALCAR entity (e.g., the ALCAR or a salt thereof or a dipeptide or salt thereof, or a tripeptide or salt thereof, comprising ALCAR) is at least 1 wt. % of the total wt. of protein components or total components in the composition (in dry form), e.g., the wt. % of the ALCAR entity is at least: 2 wt. %, 3 wt. %, or 4 wt. % of the total wt. of protein components or total components in the composition (in dry form). Optionally, ALCAR is not more than 10 wt. % of the total wt. of the protein components or total components in the composition in dry form.

In some embodiments, where the composition comprises all three BCAA entities, the NAC entity, and the ALCAR entity, the wt. ratio of the leucine amino acid entity:isoleucine amino acid entity:the valine amino acid entity:NAC entity:ALCAR entity is 10+/−15%:10+/−15%:10+/−15%:3+/−15%:1.2+/−15%, where the ratios are determined based on an equivalent amount of each amino acid in free form.

In some embodiments where the composition includes all three BCAA entities, the NAC entity, the ALCAR entity, and the creatine entity, the wt. ratio of the leucine amino acid entity:isoleucine amino acid entity:the valine amino acid entity:NAC entity:ALCAR entity:creatine entity is 10+/−15%:10+/−15%:10+/−15%:3+/−15%:1.2+/−15%:20+/−15%, where the ratios are determined based on an equivalent amount of each amino acid in free form.

In some embodiments, the composition is capable of one, two, three, four, five, six, seven, eight, nine, or all of: a) increasing TCA cycle anaplerosis or ATP production; b) protecting mitochondria from calcium influx; c) decreasing free radicals or reactive oxidative species (ROS); d) decreasing pro-inflammatory cytokines, e.g., from the activation of one or both of abnormal microglia or astrocyte; e) decreasing microglial response to pro-inflammatory signals (e.g., M1 microglia phenotype to M2 microglia phenotype); f) increasing neuronal signaling (e.g. hippocampal signaling); g) decreasing inflammation (e.g., inflammation of brain tissue); h) increasing ionic flux; i) increasing mitochondrial function; or j) decreasing synaptic dysfunction.

In some embodiments, one, two, three, four, or five amino acid entities in (a)-(c) is selected from Table 1.

In some embodiments, the composition (e.g., the Active Moiety) comprises, consists of, or consists essentially of: a) the leucine amino acid entity is chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof; b) the NAC entity is NAC or a salt thereof or a dipeptide or salt thereof, or a tripeptide or salt thereof, comprising NAC; and c) the ALCAR entity is ALCAR or a salt thereof or a dipeptide or salt thereof, or a tripeptide or salt thereof, comprising ALCAR.

Optionally, in any of the foregoing Active Moiety compositions containing an isoleucine amino acid entity, the isoleucine amino acid-entity is L-isoleucine or a salt thereof or a dipeptide or salt thereof, or tripeptide or salt thereof, comprising L-isoleucine. Optionally, in any of the foregoing Active Moiety compositions containing a valine amino acid entity, the valine amino acid-entity is L-valine or a salt thereof or a dipeptide or salt thereof, or tripeptide or salt thereof, comprising L-valine.

In some embodiments, the composition comprises, consists of, or consists essentially of: L-leucine or a salt thereof, L-isoleucine or a salt thereof, L-valine or a salt thereof, NAC or a salt thereof, and ALCAR or a salt thereof (e.g., ALCAR HCl). In some embodiments, the composition comprises, consists of, or consists essentially of: L-leucine or a salt thereof, L-isoleucine or a salt thereof, L-valine or a salt thereof, NAC or a salt thereof, ALCAR or a salt thereof (e.g., ALCAR HCl), and creatine or a salt thereof.

In some embodiments, the composition (e.g., the Active Moiety) is formulated with a pharmaceutically acceptable carrier. In some embodiments, the composition (e.g., the Active Moiety) is formulated as a dietary composition. In some embodiments, the dietary composition is chosen from a medical food, a functional food, or a supplement.

In another aspect, the invention features a method of improving one or both of neuronal function or cognitive function, comprising administering to a subject in need thereof an effective amount of a composition (e.g., an Active Moiety) of any of the aspects or embodiments disclosed herein, thereby improving one or both of neuronal function or cognitive function in the subject.

In another aspect, the invention features a method of improving a symptom of stroke chosen from one, two, three, four, five, six, seven, eight, nine, 10, 11, 12, 13, 14, 15, 16, or more (e.g., all) of: numbness, decreased balance, memory loss, facial weakness, eyelid dropping, paralysis (e.g., hemiplegia), decreased sensory sensation, decreased reflexes, tongue weakness, involuntary eye moment, a visual field defect, aphasia, increased confusion, vertigo, decreased speaking ability (e.g., apraxia), decreased walking ability, or decreased motor coordination, comprising administering to a subject in need thereof an effective amount of a composition comprising: a) a Branched Chain Amino Acid (BCAA) entity chosen from a leucine amino acid entity, an isoleucine amino acid entity, a valine amino acid entity, or a combination of two or three BCAA entities; b) a N-acetylcysteine (NAC) entity; and c) an acetyl-1-carnitine (ALCAR) entity; in which at least one amino acid entity of (a)-(c) is not provided as a peptide of more than 20 amino acid residues in length, thereby improving one, two, three, four, five, six, seven, eight, nine, 10, 11, 12, 13, 14, 15, 16, or more (e.g., all) of the symptoms.

In some embodiments, administration of the composition results in an improvement in one, two, three, four, five, six, seven, eight, nine, 10, 11, 12, 13, 14, 15, 16, or more (e.g., all) of the symptoms after a treatment period of 6 hours to 24 hours.

In another aspect, the invention features a method of improving a symptom of traumatic brain injury (TBI) chosen from one, two, three, four, five, six, seven, eight, nine, 10, 11, 12, 13, 14, 15, 16, 17, 18, or more (e.g., all) of cognitive deficits, dizziness, hearing loss, headache (e.g., frequent headache), loss of consciousness, memory loss, confusion, sleep disturbance, nausea, decreased balance, fatigue, drowsiness, blurred vision, ringing in ears, sensitivity to light, sensitivity to sound, decreased ability to concentration, mood swings, or increased anxiety, comprising administering to a subject in need thereof an effective amount of a composition comprising: a) a Branched Chain Amino Acid (BCAA) entity chosen from a leucine amino acid entity, an isoleucine amino acid entity, a valine amino acid entity, or a combination of two or three BCAA entities; b) a N-acetylcysteine (NAC) entity; and c) an acetyl-1-carnitine (ALCAR) entity; in which at least one amino acid entity of (a)-(c) is not provided as a peptide of more than 20 amino acid residues in length, thereby improving one, two, three, four, five, six, seven, eight, nine, 10, 11, 12, 13, 14, 15, 16, 17, 18, or more (e.g., all) of the symptoms.

In some embodiments, administration of the composition results in an improvement in one, two, three, four, five, six, seven, eight, nine, 10, 11, 12, 13, 14, 15, 16, 17, 18, or more (e.g., all) of the symptoms after a treatment period of 6 hours to 24 hours.

In another aspect, the invention features a method for treating or preventing neuronal injury, comprising administering to a subject in need thereof an effective amount of the composition of any of the aspects and embodiments disclosed herein, thereby treating or preventing the neuronal injury.

In some embodiments, the subject is at risk of or has TBI, e.g., mild TBI (concussion).

In some embodiments, the subject is at risk of or has stroke, e.g., ischemic stroke, such as acute ischemic stroke, or transient ischemic attack.

In some embodiments, the composition is administered orally.

### BRIEF DESCRIPTION OF THE DRAWINGS

FIG. 1 is a graph showing the effect of treatment with the amino acid compositions on the beam balance following injury in a rat model of TBI. Data are presented as mean±SEM. n=15/group.

FIG. 2 is a graph showing a NeuroCube® gait analysis of Sham surgical controls and TBI rat models administered vehicle. Cloud plots illustrate the discrimination of multivariate n-dimensional features projected onto a two-dimensional space. NeuroCube® detects at significant (p<0.05) 75% discrimination between Sham surgical controls (a) and TBI animals treated with vehicle (b).

FIG. 3 is a graph showing a NeuroCube® gait analysis of the effect of treatment with the Active Moiety compositions in the rat model of TBI. Separation between “a” and “b” clouds show discrimination between sham and TBI treated with vehicle, respectively. The “c” clouds show recovery towards Sham surgical controls.

FIG. 4 is a schematic showing the design of a clinical study of the effect of administering an amino acid composition to subjects with traumatic brain injury (TBI). Arrows indicate time points for performing neurocognition assessments, sampling for serum biomarkers, amino acid levels, and metabolite levels, and pharmacokinetic profiling of the amino acid composition.

### DETAILED DESCRIPTION

Described herein, in part, is a composition (Active Moiety) comprising amino acid entities and methods of improving one or both of neuronal function or cognitive function by administering an effective amount of the composition in a pharmaceutically acceptable formulation. The composition can be administered to treat or prevent neuronal injury (e.g., traumatic brain injury (TBI), e.g., mTBI, or stroke) in a subject in need thereof (e.g, a human who has suffered a TBI or a stroke).

The invention is based, in part, on the discovery that a designed combination of amino acid entities, exemplified herein, provided for more rapid recovery from brain injury, such as due to TBI or stroke. Rats treated to simulate brain injury were able to navigate a balance beam and recover gait more rapidly when administered compositions of the invention than a placebo.

TBI is characterized by neurological deficits and central nervous system (CNS) injuries of vascular origin. Abnormal neuronal signaling in TBI can result in one, two, three, or four of cell energy crisis, oxidative stress, cell death, or neuroinflammation. Stroke (e.g., ischemic stroke) and TBI share common mechanisms resulting in neuronal injury, including, but not limited to, one, two, three, four, five, six, seven, eight, nine, 10, 11, or more of blood brain barrier (BBB) disruption, one or both of ATP depletion or ionic imbalance, immune cell infiltration, neuroinflammation, aberrant glutamate release, one or both of perfusion or metabolic dysregulation, exitoxicity, mitochondrial damage, ROS production, protease activation, cellular damage, or cell death.

The amino acid entities and relative amounts of the amino acid entities in the compositions disclosed herein have been designed to improve or treat the complex pathophysiology of neuronal injury, e.g., TBI or stroke, that requires the complex coordination of many biological, cellular, and molecular processes. In some embodiments, without being bound to theory, the compositions disclosed herein improve neuronal function by one, two, three, or four of the following: improve the TCA cycle to increase ATP production, prevent mitochondrial dysfunction due to Ca²⁺ accumulation, decrease mitochondrial dysfunction due to Ca²⁺ accumulation, or decrease neuroinflammation, e.g., by one, two, or three of scavenging free radicals, scavenging ROS, or reducing pro-inflammatory cytokines.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to an amino acid in one or both of free form or salt form, an amino acid residue of a peptide (e.g., of a dipeptide, tripeptide, oligopeptide, or polypeptide), a derivative of an amino acid, a precursor of an amino acid, or a metabolite of an amino acid.

As used herein the term “XXX amino acid entity” refers to an amino acid entity that if a free amino acid, comprises free XXX or XXX in salt form; if a peptide, refers to a peptide (e.g., a dipeptide or a tripeptide) comprising an XXX residue; if a derivative, refers to a derivative of XXX; if a precursor, refers to a precursor of XXX; and if a metabolite, refers to a XXX metabolite (Table 1).

TABLE 1
Amino acid entities include amino acids, precursors, metabolites,
and derivatives of the compositions described herein.

| | Exemplary | | | |
|---|---|---|---|---|
| | Amino Acid | Precursors | Metabolites | Derivatives |
| Leucine | L-Leucine | Oxo-leucine | HMB (beta- | N-Acetyl-Leucine |
| | | (Alpha- | hydroxy-beta- | |
| | | ketoisocaproate | methybutyrate); | |
| | | (KIC)) | Oxo-leucine; | |
| | | | Isovaleryl-CoA | |
| Isoleucine | L-Isoleucine | 2-Oxo-3- | 2-Oxo-3-methyl- | N-Acetyl-Isoleucine |
| | | methyl-valerate | valerate; | |
| | | (Alpha-keto- | Methylbutyrl- | |
| | | beta- | CoA | |
| | | methylvaleric | | |
| | | acid (KMV)); | | |
| | | Threonine | | |
| Valine | L-Valine | 2-Oxo-valerate | Isobutryl-CoA | N-Acetyl-Valine |
| | | (Alpha- | | |
| | | ketoisovalerate | | |
| | | (KIV)), | | |
| NAC | N- | Serine; | Glutathione; | L-Cysteine; Cystine; |
| | Acetylcysteine | Acetylserine; | Cystathionine; | Cysteamine |
| | | Cystathionine; | Homocysteine; | |
| | | | Methionine | |
| ALCAR | Acetyl-L- | Lysine, | Carnitine, O- | Citrate, succinate, C3- |
| | Carnitine | Trimethyllysine, | acyl-carnitine, | carnitine, C5-carnitine, C4- |
| | | Trimethyl-3- | Acyl-CoA | dicarboxylcarnitine, C6- |
| | | OH-lysine | | carnitine, C8-carnitine, |
| | | | | C12:1 acylcarnitine, C14 |
| | | | | acylcarnitine, C14:1 |
| | | | | acylcarnitine, C16 |
| | | | | acylcarnitine, C18:2 |
| | | | | acylcarnitine, C18:1 |
| | | | | acylcarnitine, C18 |
| | | | | acylcarnitine |
| Creatine | Creatine | Guanidoacetate, | Creatinine, | |
| | | L-Glycine; L- | Phosphocreatine | |
| | | arginine | | |

For example, where XXX is leucine (L), then leucine amino acid entity refers to free L or L in salt form, a peptide (e.g., a dipeptide or a tripeptide) comprising a L residue, a L derivative, a L precursor, or a metabolite of L; where XXX is isoleucine (I), then isoleucine amino acid entity refers to free I or I in salt form, a peptide (e.g., a dipeptide or a tripeptide) comprising a I residue, a I derivative, a I precursor, or a metabolite of I; where XXX is valine (V), then valine amino acid entity refers to free V or V in salt form, a peptide (e.g., a dipeptide or a tripeptide) comprising a V residue, a V derivative, a V precursor, or a metabolite of V; where XXX is N-acetylcysteine (NAC), then NAC entity refers to free NAC or NAC in salt form, a peptide (e.g., a dipeptide or a tripeptide) comprising a NAC residue, a NAC derivative, a NAC precursor, or a metabolite of NAC; where XXX is acetyl-1-carnitine (ALCAR), then ALCAR entity refers to free ALCAR or ALCAR in salt form, a peptide (e.g., a dipeptide or a tripeptide) comprising a ALCAR residue, a ALCAR derivative, a ALCAR precursor, or a metabolite of ALCAR; where XXX is creatine (CR), then CR amino acid entity refers to free CR or CR in salt form, a peptide (e.g., a dipeptide or a tripeptide) comprising a CR residue, a CR derivative, a CR precursor, or a metabolite of CR.

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids, as well as natural and unnatural amino acids. Unless otherwise indicated, amino acids referred to herein are L-isomers of amino acids.

As used herein, the term “Active Moiety” means a combination of four or more amino acid entities that, in aggregate, have the ability to have a physiological effect as described herein, e.g., improving one or both of neuronal function or cognitive function. For example, an Active Moiety can treat or prevent neuronal injury. An Active Moiety of the invention can contain other biologically active ingredients. In some examples, the Active Moiety comprises a defined combination of four or more amino acid entities, as set out in detail below. In other embodiments, the Active Moiety consists of a defined combination of four or more amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally this disclosure will provide grams of amino acid entity in a dosage form, weight percent of an amino acid entity relative to the weight of the composition, i.e., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

The term “effective amount” as used herein means an amount of an amino acid, or pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “equivalent amount” of an amino acid entity is an amount that yields, physiologically, the same activity as that amount of the corresponding free amino acid for the amino acid entity.

A “pharmaceutical composition” described herein comprises at least one “Active Moiety” and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions”.

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “protein component,” as used herein, refers to a peptide (e.g., a polypeptide or an oligopeptide), a fragment thereof, a degraded peptide, an amino acid entity or a free amino acid. A protein component includes an amino acid in free form or salt form, a dipeptide of an amino acid, a tripeptide of an amino acid, a derivative of an amino acid, a precursor of an amino acid, or a metabolite of an amino acid. Exemplary protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A composition, formulation or product is “therapeutic” if it provides a beneficial clinical effect. A beneficial clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g, a capsule shell, for example), and apportioned into a particular dose (e.g., in multiple stick packs).

As used herein, the terms “treat,” “treating,” or “treatment” of liver disease or disorder or muscle wasting refers to ameliorating neuronal injury (e.g., slowing, arresting, or reducing the development of neuronal injury at least one of the clinical symptoms thereof); alleviating or ameliorating at least one physical parameter including those which may not be discernible by the patient; and/or preventing or delaying the onset or development or progression of neuronal injury.

### Compositions Comprising Amino Acid Entities

The composition of the invention as described herein (e.g., an Active Moiety) comprises amino acid entities, e.g., the amino acid entities shown in Table 1.

In some embodiments, the leucine amino acid entity is chosen from Table 1, e.g., the L-amino acid entity is chosen from L-leucine, β-hydroxy-β-methylbutyrate (HMB), oxo-leucine (alpha-ketoisocaproate (KIC)), isovaleryl-CoA, n-acetylleucine, or a combination thereof.

In some embodiments, the NAC entity is chosen from Table 1, e.g., the NAC-entity is chosen from NAC, serine, acetylserine, cystathionine, glutathione, homocysteine, methionine, L-cysteine, cysteamine, cystine, or a combination thereof. In some embodiments, the NAC-entity is chosen from NAC, acetylserine, cystathionine, glutathione, homocysteine, cysteamine, or a combination thereof.

In some embodiments, the ALCAR entity is chosen from Table 1, e.g., the ALCAR-entity is chosen from ALCAR, L-lysine, trimethyllysine, trimethyl-3-OH-lysine, L-carnitine, O-acyl-carnitine, acetyl-CoA, citrate, succinate, C3-carnitine, C5-carnitine, C4-dicarboxylcarnitine, C6-carnitine, C8-carnitine, C12:1 acylcarnitine, C14 acylcarnitine, C14:1 acylcarnitine, C16 acylcarnitine, C18:2 acylcarnitine, C18:1 acylcarnitine, C18 acylcarnitine, or a combination thereof. In some embodiments, the ALCAR entity is chosen from ALCAR, trimethyllysine, trimethyl-3-OH-lysine, O-acyl-carnitine, acetyl-CoA, citrate, succinate, C3-carnitine, C5-carnitine, C4-dicarboxylcarnitine, C6-carnitine, C8-carnitine, C12:1 acylcarnitine, C14 acylcarnitine, C14:1 acylcarnitine, C16 acylcarnitine, C18:2 acylcarnitine, C18:1 acylcarnitine, C18 acylcarnitine, or a combination thereof.

In some embodiments, the isoleucine amino acid entity is chosen from Table 1, e.g., the I-amino acid entity is chosen from L-isoleucine, 2-oxo-3-methyl-valerate (alpha-keto-beta-methylvaleric acid (KMV)), threonine, 2-oxo-3-methyl-valerate, methylbutyrl-CoA, N-acetyl-isoleucine, or a combination thereof.

In some embodiments, the valine amino acid entity is chosen from Table 1, e.g., the V-amino acid entity is chosen from L-valine, 2-oxo-valerate 2-oxo-valerate (alpha-ketoisovalerate (KIV)), isobutryl-CoA, N-acetyl-valine, or a combination thereof.

In some embodiments, the creatine entity is chosen from Table 1, e.g., the creatine entity is chosen from creatine, guanidoacetate, glycine, L-arginine, creatinine, phosphocreatine, or a combination thereof. In some embodiments, the creatine entity is chosen from creatine, guanidoacetate, creatinine, phosphocreatine, or a combination thereof.

In some embodiments, the composition comprises a leucine amino acid entity, an isoleucine amino acid entity, a valine amino acid entity, and a NAC entity. In some embodiments, the composition further comprises one or both of an ALCAR entity or a creatine entity.

In some embodiments, the composition comprises, consists of, or consists essentially of:
a) a Branched Chain Amino Acid (BCAA) entity chosen from a leucine amino acid entity, an isoleucine amino acid entity, a valine amino acid entity, or a combination of two or three BCAA entities; b) a N-acetylcysteine (NAC) entity; and c) a creatine entity;
in which at least one amino acid entity of (a)-(c) is not provided as a peptide of more than 20 amino acid residues in length.

In some embodiments, this composition further comprises (d) an ALCAR entity. In some embodiments, the ALCAR entity is not provided as a peptide of more than 20 amino acid residues in length. In some embodiments, the ALCAR entity is selected from Table 1.

In some embodiments, the composition comprises, consists of, or consists essentially of: a) the leucine amino acid entity is chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof; b) the isoleucine amino acid entity is L-isoleucine or a salt thereof or a dipeptide or salt thereof, or tripeptide or salt thereof, comprising L-isoleucine; c) the valine amino acid entity is L-valine or a salt thereof or a dipeptide or salt thereof, or tripeptide or salt thereof, comprising L-valine; d) the NAC entity is NAC or a salt thereof or a dipeptide or salt thereof, or a tripeptide or salt thereof, comprising NAC; and e) the ALCAR entity is ALCAR or a salt thereof or a dipeptide or salt thereof, or a tripeptide or salt thereof, comprising ALCAR.

In some embodiments, the composition comprises, consists of, or consists essentially of: L-leucine or a salt thereof, L-isoleucine or a salt thereof, L-valine or a salt thereof, NAC or a salt thereof, and creatine or a salt thereof.

In some embodiments, one, two, or three of (a) a leucine amino acid entity, an isoleucine amino acid entity, or a valine amino acid entity is in free amino acid form. In some embodiments, one, two, or three of (a) a leucine amino acid entity, an isoleucine amino acid entity, a valine amino acid entity is in salt amino acid form.

In some embodiments, NAC is in free amino acid form. In some embodiments, NAC is in salt amino acid form. In some embodiments, ALCAR is in free amino acid form. In some embodiments, ALCAR is in salt amino acid form (e.g., ALCAR HCl). In some embodiments, creatine is in free amino acid form. In some embodiments, creatine is in salt amino acid form.

In some embodiments, at least: 35 wt. %, 40 wt. %, 42 wt. %, 45 wt. %, 50 wt. %, 55 wt. %, 60 wt. %, or more, of the total wt. of the composition (e.g., in dry form) is one, two, three, four, or five amino acid entities of (a)-(c) or one, two, three, four, five, or six amino acid entities of (a)-(d) in free amino acid form.

In some embodiments, one, two, three, four, or five amino acid entities of (a)-(c) or one, two, three, four, five, or six amino acid entities of (a)-(d) are in free amino acid form in the composition, e.g., at least: 42 wt. %, 75 wt. %, or 90 wt. % of the total wt. of protein components or total components is one, two, three, four, or five amino acid entities of (a)-(c) or one, two, three, four, five, or six amino acid entities of (a)-(d) in free amino acid form in the composition (e.g., in dry form).

In some embodiments, one, two, three, four, or five amino acid entities of (a)-(c) or one, two, three, four, five, or six amino acid entities of (a)-(d) are in salt form in the composition, e.g., at least: 0.01 wt. %, 0.1 wt. %, 0.5 wt. %, 1 wt. %, 5 wt. %, or 10 wt. %, or more of the total wt. of protein components or total components is one, two, three, four, or five amino acid entities of (a)-(c) or one, two, three, four, five, or six amino acid entities of (a)-(d) in salt form in the composition (e.g., in dry form).

In some embodiments, one, two, three, four, or five amino acid entities of (a)-(c) or one, two, three, four, five, or six amino acid entities of (a)-(d) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of protein components or total components of the composition.

In some embodiments, one, two, or three of the leucine amino acid entity, the isoleucine amino acid entity, or the valine amino acid entity is provided as part of a dipeptide (e.g., a homodipeptide or heterodipeptide) or salt thereof. In some embodiments, the leucine amino acid entity is Ala-Leu. In some embodiments, one, two, or three of the leucine amino acid entity, the isoleucine amino acid entity, or the valine amino acid entity is provided as part of a tripeptide (e.g., a homotripeptide or heterotripeptide) or salt thereof. In some embodiments, one, two, or three of the NAC entity, the ALCAR entity, or the creatine entity is provided as part of a dipeptide (e.g., a heterodipeptide) or salt thereof. In some embodiments, one, two, or three of the NAC entity, the ALCAR entity, or the creatine entity is provided as part of a tripeptide (e.g., a heterotripeptide) or salt thereof.

In some embodiments the composition (e.g., the Active Moiety) is capable of decreasing, or decreases, inflammation (e.g., neuroinflammation) by at least 20%, 30%, or 35%, as detected using an assay of IL-6, e.g., in microglial cells, e.g., using an antibody-based detection assay, e.g., an ELISA, e.g., as described in Example 1, e.g., relative to a reference composition (e.g., an amino acid composition comprising L-leucine, L-isoleucine, and L-valine; NAC; creatine; or ALCAR).

In some embodiments the composition (e.g., the Active Moiety) is capable of decreasing, or decreases, inflammation (e.g., neuroinflammation) by at least 10%, 25%, 30%, 40%, or 50%, as detected using an assay of TNFα, e.g., in microglial cells, e.g., using an antibody-based detection assay, e.g., an ELISA, e.g., as described in Example 1, e.g., relative to a reference composition (e.g., an amino acid composition comprising L-leucine, L-isoleucine, L-valine, and NAC; L-leucine, L-isoleucine, and L-valine; NAC; creatine; or ALCAR).

The present disclosure provides composition including free amino acids, in which the amino acids include leucine, isoleucine, valine, and N-acetylcysteine. In some embodiments, leucine, isoleucine, and valine are present in the composition at a weight ratio of about 1:1:1 or about 15:6:18. In certain embodiments, leucine, isoleucine, valine, and N-acetylcysteine are present in a weight ratio of about 15:6:18:6 or about 15:6:18:8.

In some embodiments, the composition further comprises creatine or L-carnitine. In some embodiments, the composition further comprises creatine and L-carnitine. In some embodiments, the L-carnitine is acetyl L-carnitine.

In an embodiment, the composition consists of leucine, isoleucine, valine, N-acetylcysteine, L-carnitine, and one or more pharmaceutically acceptable excipients. In an embodiment, the composition consists of leucine, isoleucine, valine, N-acetylcysteine, creatine, and one or more pharmaceutically acceptable excipients. In an embodiment, the composition consists of leucine, isoleucine, valine, N-acetylcysteine, creatine, L-carnitine, and one or more pharmaceutically acceptable excipients.

In some embodiments, the composition further comprises one, two, three, or four of L-arginine, L-glutamine, glycine, or L-serine. In some embodiments, the composition further comprises an inducible nitric oxide synthase (iNOS) inhibitor, e.g., ronopterin (VAS203).

i. Amounts

An exemplary composition (e.g., an Active Moiety) can include 1.67 g of leucine or the equivalent amount of a leucine amino acid entity, 1.67 g of isoleucine or the equivalent amount of an isoleucine amino acid entity, 1.67 g of valine or the equivalent amount of a valine amino acid entity, 0.5 g of NAC or the equivalent amount of a NAC entity, and 0.21 g of ALCAR or the equivalent amount of an ALCAR entity (see, e.g., g/stick pack in Table 2).

TABLE 2
Exemplary composition comprising amino acids (e.g., an Active Moiety).

| | Stick Pack | Dose | Daily Dose | Wt. | |
|---|---|---|---|---|---|
| Amino Acid | (g) | (g) | (g) | Ratio | Wt. % |
| L-leucine | 1.67 | 5 | 10 | 10 | 29.1 (29.2) |
| L-isoleucine | 1.67 | 5 | 10 | 10 | 29.1 (29.2) |
| L-valine | 1.67 | 5 | 10 | 10 | 29.1 (29.2) |
| N-acetylcysteine | 0.5 | 1.5 | 3 | 3 | 8.7 (8.8) |
| Acetyl-l-carnitine | 0.24 (0.21 | 0.7 (0.6 | 1.4 (1.2 | 1.4 (1.2 | 4.1 (3.5 |
| HCl* | ALCAR) | ALCAR) | ALCAR) | ALCAR) | ALCAR) |
| Total amino acids | 5.74 | 17.2 | 34.4 | | |

*Amounts (in g) of acetyl-l-carnitine HCl are provided in salt form with the free form amount of acetyl-l-carnitine in parentheses.

In some embodiments, the composition includes 1.67 g+/−20% of leucine or the equivalent amount of a leucine amino acid entity, 1.67 g+/−20% of isoleucine or the equivalent amount of an isoleucine amino acid entity, 1.67 g+/−20% of valine or the equivalent amount of a valine amino acid entity, 0.5 g+/−20% of NAC or the equivalent amount of a NAC entity, and 0.21 g+/−20% of ALCAR or the equivalent amount of an ALCAR entity.

In some embodiments, the composition includes 1.67 g+/−15% of leucine or the equivalent amount of a leucine amino acid entity, 1.67 g+/−15% of isoleucine or the equivalent amount of an isoleucine amino acid entity, 1.67 g+/−15% of valine or the equivalent amount of a valine amino acid entity, 0.5 g+/−15% of NAC or the equivalent amount of a NAC entity, and 0.21 g+/−15% of ALCAR or the equivalent amount of an ALCAR entity.

In some embodiments, the composition includes 1.67 g+/−10% of leucine or the equivalent amount of a leucine amino acid entity, 1.67 g+/−10% of isoleucine or the equivalent amount of an isoleucine amino acid entity, 1.67 g+/−10% of valine or the equivalent amount of a valine amino acid entity, 0.5 g+/−10% of NAC or the equivalent amount of a NAC entity, and 0.21 g+/−10% of ALCAR or the equivalent amount of an ALCAR entity.

In some embodiments, the composition includes 1.67 g+/−5% of leucine or the equivalent amount of a leucine amino acid entity, 1.67 g+/−5% of isoleucine or the equivalent amount of an isoleucine amino acid entity, 1.67 g+/−5% of valine or the equivalent amount of a valine amino acid entity, 0.5 g+/−5% of NAC or the equivalent amount of a NAC entity, and 0.21 g+/−15% of ALCAR or the equivalent amount of an ALCAR entity.

An exemplary composition (e.g., an Active Moiety) can include 1.25 g of leucine or the equivalent amount of a leucine amino acid entity, 1.25 g of isoleucine or the equivalent amount of an isoleucine amino acid entity, 1.25 g of valine or the equivalent amount of a valine amino acid entity, 0.38 g of NAC or the equivalent amount of a NAC entity, 0.15 g of ALCAR or the equivalent amount of an ALCAR entity, and 2.5 g of creatine or the equivalent amount of a creatine entity (see, e.g., g/stick pack in Table 3).

TABLE 3
Exemplary composition comprising amino acids (e.g., an Active Moiety).

| | Stick Pack | Dose | Daily Dose | Wt. | |
|---|---|---|---|---|---|
| Amino Acid | (g) | (g) | (g) | Ratio | Wt. % |
| L-leucine | 1.25 | 5 | 10 | 10 | 18.4 (18.5) |
| L-isoleucine | 1.25 | 5 | 10 | 10 | 18.4 (18.5) |
| L-valine | 1.25 | 5 | 10 | 10 | 18.4 (18.5) |
| N-acetylcysteine | 0.38 | 1.5 | 3 | 3 | 5.5 (5.5) |
| Acetyl-l-carnitine | 0.18 (0.15 | 0.7 (0.6 | 1.4 (1.2 | 1.4 (1.2 | 2.6 (2.2 |
| HCl* | ALCAR) | ALCAR) | ALCAR) | ALCAR) | ALCAR) |
| Creatine | 2.5 | 10 | 20 | 20 | 36.8 (36.9) |
| Total amino acids | 6.80 | 27.2 | 54.4 | | 100 |

*Amounts (in g) of acetyl-l-carnitine HCl are provided in salt form with the free form amount of acetyl-l-carnitine in parentheses.

In some embodiments, the composition includes 1.25 g+/−20% of leucine or the equivalent amount of a leucine amino acid entity, 1.25 g+/−20% of isoleucine or the equivalent amount of an isoleucine amino acid entity, 1.25 g+/−20% of valine or the equivalent amount of a valine amino acid entity, 0.38 g+/−20% of NAC or the equivalent amount of a NAC entity, 0.15 g+/−20% of ALCAR or the equivalent amount of an ALCAR entity, and 2.5 g+/−20% of creatine or the equivalent amount of a creatine entity.

In some embodiments, the composition includes 1.25 g+/−15% of leucine or the equivalent amount of a leucine amino acid entity, 1.25 g+/−15% of isoleucine or the equivalent amount of an isoleucine amino acid entity, 1.25 g+/−15% of valine or the equivalent amount of a valine amino acid entity, 0.38 g+/−15% of NAC or the equivalent amount of a NAC entity, 0.15 g+/−15% of ALCAR or the equivalent amount of an ALCAR entity, and 2.5 g+/−15% of creatine or the equivalent amount of a creatine entity.

In some embodiments, the composition includes 1.25 g+/−10% of leucine or the equivalent amount of a leucine amino acid entity, 1.25 g+/−10% of isoleucine or the equivalent amount of an isoleucine amino acid entity, 1.25 g+/−10% of valine or the equivalent amount of a valine amino acid entity, 0.38 g+/−10% of NAC or the equivalent amount of a NAC entity, 0.15 g+/−10% of ALCAR or the equivalent amount of an ALCAR entity, and 2.5 g+/−10% of creatine or the equivalent amount of a creatine entity.

In some embodiments, the composition includes 1.25 g+/−5% of leucine or the equivalent amount of a leucine amino acid entity, 1.25 g+/−5% of isoleucine or the equivalent amount of an isoleucine amino acid entity, 1.25 g+/−5% of valine or the equivalent amount of a valine amino acid entity, 0.38 g+/−5% of NAC or the equivalent amount of a NAC entity, 0.15 g+/−5% of ALCAR or the equivalent amount of an ALCAR entity, and 2.5 g+/−5% of creatine or the equivalent amount of a creatine entity.

ii. Ratios

In some embodiments, the wt. ratio of the BCAA entity or BCAA entities:NAC entity:ALCAR entity is 10+/−20%:3+/−20%:1.2+/−20%, where the ratios are determined based on an equivalent amount of each amino acid in free form. In some embodiments, the wt. ratio of the BCAA entity or BCAA entities:NAC entity:ALCAR entity is 10+/−15%:3+/−15%:1.2+/−15%, where the ratios are determined based on an equivalent amount of each amino acid in free form. In some embodiments, the wt. ratio of the BCAA entity or BCAA entities:NAC entity:ALCAR entity is 10+/−10%:3+/−10%:1.2+/−10%, where the ratios are determined based on an equivalent amount of each amino acid in free form. In some embodiments, the wt. ratio of the BCAA entity or BCAA entities:NAC entity:ALCAR entity is 10+/−5%:3+/−5%:1.2+/−5%, where the ratios are determined based on an equivalent amount of each amino acid in free form.

In some embodiments, the wt. ratio of the BCAA entity or BCAA entities:NAC entity:creatine entity is 10+/−20%:3+/−20%:20+/−20%, where the ratios are determined based on an equivalent amount of each amino acid in free form. In some embodiments, the wt. ratio of the BCAA entity or BCAA entities:NAC entity:creatine entity is 10+/−15%:3+/−15%:20+/−15%, where the ratios are determined based on an equivalent amount of each amino acid in free form. In some embodiments, the wt. ratio of the BCAA entity or BCAA entities:NAC entity:creatine entity is 10+/−10%:3+/−10%:20+/−10%, where the ratios are determined based on an equivalent amount of each amino acid in free form. In some embodiments, the wt. ratio of the BCAA entity or BCAA entities:NAC entity:creatine entity is 10+/−5%:3+/−5%:20+/−5%, where the ratios are determined based on an equivalent amount of each amino acid in free form.

In some embodiments, the wt. ratio of the leucine amino acid entity:isoleucine amino acid entity:the valine amino acid entity is 10+/−20%:10+/−20%:10+/−20%, where the ratios are determined based on an equivalent amount of each amino acid in free form. In some embodiments, the wt. ratio of the leucine amino acid entity:isoleucine amino acid entity:the valine amino acid entity is 10+/−15%:10+/−15%:10+/−15%, where the ratios are determined based on an equivalent amount of each amino acid in free form. In some embodiments, the wt. ratio of the leucine amino acid entity:isoleucine amino acid entity:the valine amino acid entity is 10+/−10%:10+/−10%:10+/−10%, where the ratios are determined based on an equivalent amount of each amino acid in free form. In some embodiments, the wt. ratio of the leucine amino acid entity:isoleucine amino acid entity:the valine amino acid entity is 10+/−5%:10+/−5%:10+/−5%, where the ratios are determined based on an equivalent amount of each amino acid in free form.

In some embodiments, the wt. ratio of the leucine amino acid entity:isoleucine amino acid entity:the valine amino acid entity is 15+/−20%:6+/−20%:18+/−20%, where the ratios are determined based on an equivalent amount of each amino acid in free form. In some embodiments, the wt. ratio of the leucine amino acid entity:isoleucine amino acid entity:the valine amino acid entity is 15+/−15%:6+/−15%:18+/−15%, where the ratios are determined based on an equivalent amount of each amino acid in free form. In some embodiments, the wt. ratio of the leucine amino acid entity:isoleucine amino acid entity:the valine amino acid entity is 15+/−10%:6+/−10%:18+/−10%, where the ratios are determined based on an equivalent amount of each amino acid in free form. In some embodiments, the wt. ratio of the leucine amino acid entity:isoleucine amino acid entity:the valine amino acid entity is 15+/−5%:6+/−5%:18+/−5%, where the ratios are determined based on an equivalent amount of each amino acid in free form.

In some embodiments, the wt. ratio of the leucine amino acid entity:isoleucine amino acid entity:the valine amino acid entity:NAC entity is 10+/−20%:10+/−20%:10+/−20%:3+/−20%, where the ratios are determined based on an equivalent amount of each amino acid in free form. In some embodiments, the wt. ratio of the leucine amino acid entity:isoleucine amino acid entity:the valine amino acid entity:NAC entity is 10+/−15%:10+/−15%:10+/−15%:3+/−15%, where the ratios are determined based on an equivalent amount of each amino acid in free form. In some embodiments, the wt. ratio of the leucine amino acid entity:isoleucine amino acid entity:the valine amino acid entity:NAC entity is 10+/−10%:10+/−10%:10+/−10%:3+/−10%, where the ratios are determined based on an equivalent amount of each amino acid in free form. In some embodiments, the wt. ratio of the leucine amino acid entity:isoleucine amino acid entity:the valine amino acid entity:NAC entity is 10+/−5%:10+/−5%:10+/−5%:3+/−5%, where the ratios are determined based on an equivalent amount of each amino acid in free form.

In some embodiments, the wt. ratio of the leucine amino acid entity:isoleucine amino acid entity:the valine amino acid entity:NAC entity is 10+/−20%:10+/−20%:10+/−20%:4+/−20%, where the ratios are determined based on an equivalent amount of each amino acid in free form. In some embodiments, the wt. ratio of the leucine amino acid entity:isoleucine amino acid entity:the valine amino acid entity:NAC entity is 10+/−15%:10+/−15%:10+/−15%:4+/−15%, where the ratios are determined based on an equivalent amount of each amino acid in free form. In some embodiments, the wt. ratio of the leucine amino acid entity:isoleucine amino acid entity:the valine amino acid entity:NAC entity is 10+/−10%:10+/−10%:10+/−10%:4+/−10%, where the ratios are determined based on an equivalent amount of each amino acid in free form. In some embodiments, the wt. ratio of the leucine amino acid entity:isoleucine amino acid entity:the valine amino acid entity:NAC entity is 10+/−5%:10+/−5%:10+/−5%:4+/−5%, where the ratios are determined based on an equivalent amount of each amino acid in free form.

In some embodiments, the wt. ratio of the leucine amino acid entity:isoleucine amino acid entity:the valine amino acid entity:NAC entity is 15+/−20%:6+/−20%:18+/−20%:6+/−20%, where the ratios are determined based on an equivalent amount of each amino acid in free form. In some embodiments, the wt. ratio of the leucine amino acid entity:isoleucine amino acid entity:the valine amino acid entity:NAC entity is 15+/−15%:6+/−15%:18+/−15%:6+/−15%, where the ratios are determined based on an equivalent amount of each amino acid in free form. In some embodiments, the wt. ratio of the leucine amino acid entity:isoleucine amino acid entity:the valine amino acid entity:NAC entity is 15+/−10%:6+/−10%:18+/−10%:6+/−10%, where the ratios are determined based on an equivalent amount of each amino acid in free form. In some embodiments, the wt. ratio of the leucine amino acid entity:isoleucine amino acid entity:the valine amino acid entity:NAC entity is 15+/−5%:6+/−5%:18+/−5%:6+/−5%, where the ratios are determined based on an equivalent amount of each amino acid in free form.

In some embodiments, the wt. ratio of the leucine amino acid entity:isoleucine amino acid entity:the valine amino acid entity:NAC entity is 15+/−20%:6+/−20%:18+/−20%:8+/−20%, where the ratios are determined based on an equivalent amount of each amino acid in free form. In some embodiments, the wt. ratio of the leucine amino acid entity:isoleucine amino acid entity:the valine amino acid entity:NAC entity is 15+/−15%:6+/−15%:18+/−15%:8+/−15%, where the ratios are determined based on an equivalent amount of each amino acid in free form. In some embodiments, the wt. ratio of the leucine amino acid entity:isoleucine amino acid entity:the valine amino acid entity:NAC entity is 15+/−10%:6+/−10%:18+/−10%:8+/−10%, where the ratios are determined based on an equivalent amount of each amino acid in free form. In some embodiments, the wt. ratio of the leucine amino acid entity:isoleucine amino acid entity:the valine amino acid entity:NAC entity is 15+/−5%:6+/−5%:18+/−5%:8+/−5%, where the ratios are determined based on an equivalent amount of each amino acid in free form.

In some embodiments, the wt. ratio of the leucine amino acid entity:isoleucine amino acid entity:the valine amino acid entity:NAC entity:ALCAR entity is 10+/−20%:10+/−20%:10+/−20%:3+/−20%:1.2+/−20%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity is 10+/−15%:10+/−15%:10+/−15%:3+/−15%:1.2+/−15%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity is 10+/−10%:10+/−10%:10+/−10%:3+/−10%:1.2+/−10%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity is 10+/−5%:10+/−5%:10+/−5%:3+/−5%:1.2+/−5%, where the ratios are determined based on an equivalent amount of each amino acid in free form.

In some embodiments, the wt. ratio of the leucine amino acid entity:isoleucine amino acid entity:the valine amino acid entity:NAC entity:creatine entity is 10+/−20%:10+/−20%:10+/−20%:3+/−20%:20+/−20%, where the ratios are determined based on an equivalent amount of each amino acid in free form. In some embodiments, the wt. ratio of the leucine amino acid entity:isoleucine amino acid entity:the valine amino acid entity:NAC entity:creatine entity is 10+/−15%:10+/−15%:10+/−15%:3+/−15%:20+/−15%, where the ratios are determined based on an equivalent amount of each amino acid in free form. In some embodiments, the wt. ratio of the leucine amino acid entity:isoleucine amino acid entity:the valine amino acid entity:NAC entity:creatine entity is 10+/−10%:10+/−10%:10+/−10%:3+/−10%:20+/−10%, where the ratios are determined based on an equivalent amount of each amino acid in free form. In some embodiments, the wt. ratio of the leucine amino acid entity:isoleucine amino acid entity:the valine amino acid entity:NAC entity:creatine entity is 10+/−5%:10+/−5%:10+/−5%:3+/−5%:20+/−5%, where the ratios are determined based on an equivalent amount of each amino acid in free form.

In some embodiments, the wt. ratio of the leucine amino acid entity:isoleucine amino acid entity:the valine amino acid entity:NAC entity:ALCAR entity:creatine entity is 10+/−20%:10+/−20%:10+/−20%:3+/−20%:1.2+/−20%:20+/−20%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity:creatine entity is 10+/−15%:10+/−15%:10+/−15%:3+/−15%:1.2+/−15%:20+/−15%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity:creatine entity is 10+/−20%:10+/−10%:10+/−10%:3+/−10% 1.2+/−10%:20+/−10%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity:creatine entity is 10+/−5%:10+/−5%:10+/−5%:3+/−5%:1.2+/−5%:20+/−5%, where the ratios are determined based on an equivalent amount of each amino acid in free form.

In some embodiments, the wt. ratio of the leucine amino acid entity:isoleucine amino acid entity:the valine amino acid entity:NAC entity:ALCAR entity:creatine entity is 10+/−20%:10+/−20%:10+/−20%:4+/−20%:1.2+/−20%:20+/−20%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity:creatine entity is 10+/−15%:10+/−15%:10+/−15%:4+/−15%:1.2+/−15%:20+/−15%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity:creatine entity is 10+/−10%:10+/−10%:10+/−10%:4+/−10% 1.2+/−10%:20+/−10%, where the ratios are determined based on an equivalent amount of each amino acid in free form. In some embodiments, the wt. ratio of the leucine amino acid entity:isoleucine amino acid entity:the valine amino acid entity:NAC entity:ALCAR entity:creatine entity is 10+/−5%:10+/−5%:10+/−5%:4+/−5%:1.2+/−5%:20+/−5%, where the ratios are determined based on an equivalent amount of each amino acid in free form.

iii. Relationships of Amino Acid Entities

In some embodiments, the wt. % of the BCAA entity or combination of two or three BCAA enties (e.g., one, two, or three of a leucine amino acid entity, an isoleucine amino acid entity, or a valine amino acid entity) is at least 50 wt. % of the total wt. of amino acid entities or total components in the composition (e.g., in dry form), e.g., the wt. % of the BCAA entity or combination of two or three BCAA enties is at least: 55 wt. %, 60 wt. %, 65 wt. %, 70 wt. %, 75 wt. %, 80 wt. %, 85 wt. %, or more of the total wt. of amino acid entities or total components in the composition (e.g., in dry form), e.g., but not more than 95 wt. % of the total wt. of amino acid entities or total components in the composition (e.g., in dry form).

In some embodiments, the wt. % of the BCAA entity or combination of two or three BCAA enties (e.g., one, two, or three of a leucine amino acid entity, an isoleucine amino acid entity, or a valine amino acid entity), the NAC entity, and the creatine entity in combination is at least: 60 wt. %, 70 wt. %, 80 wt. %, 90 wt. %, 95 wt. %, or more of amino acid entities or total components in the composition (e.g., in dry form).

In some embodiments, the wt. % of the BCAA entity or combination of two or three BCAA enties (e.g., one, two, or three of a leucine amino acid entity, an isoleucine amino acid entity, or a valine amino acid entity) in the composition (e.g., in dry form) is greater than the wt. % of one or both of the ALCAR entity or the NAC entity, e.g., the wt. % of the BCAA entity or combination of two or three BCAA enties in the composition (e.g., in dry form) is at least 40% greater than the wt. % of one or both of the ALCAR entity or the NAC entity, e.g., the wt. % of the BCAA entity or combination of two or three BCAA enties in the composition (e.g., in dry form) is at least 50%, 60%, or 70% greater than the wt. % of one or both of the ALCAR entity or the NAC entity.

In some embodiments, the wt. % of one or both of the leucine amino acid entity or the isoleucine amino acid entity is equal to wt. % the valine amino acid entity in the composition.

In some embodiments, the wt. % of the NAC entity in the composition (e.g., in dry form) is greater than the wt. % of the ALCAR entity, e.g., the wt. % of the NAC entity in the composition (e.g., in dry form) is at least 25% greater than the wt. % of the ALCAR entity, e.g., the wt. % of the NAC entity is at least 35%, 40%, or 50% greater than the wt. % of the ALCAR entity.

In some embodiments, the wt. % of the NAC entity and the ALCAR entity in combination is at least 5 wt. % of the amino acid entities or total components in the composition (e.g., in dry form), e.g., the wt. % of the NAC entity and the ALCAR entity in combination is at least: 6 wt. %, 7 wt. %, 8 wt. %, 9 wt. %, 10 wt. %, 12 wt. %, or more of the amino acid entities or total components in the composition (e.g., in dry form), but not more than 30 wt. % of the protein components or total components in the composition (e.g., in dry form).

In some embodiments, the creatine entity is present at a greater wt. % than any other amino acid entity or non-protein component in the composition (e.g., in dry form). In some embodiments, the wt. % of the creatine entity is at least 15 wt. % of the amino acid entities or total components in the composition (e.g., in dry form), e.g., the wt. % of the creatine entity is at least: 20 wt. %, 25 wt. %, 30 wt. %, 35 wt. %, or more of amino acid entities or total components in the composition (e.g., in dry form), but not more than 60 wt. % of the amino acid entities or total components in the composition (e.g., in dry form).

In some embodiments, CR or a salt thereof or a dipeptide or salt thereof, or a tripeptide or salt thereof, comprising CR is present at a greater wt. % than any other amino acid entity or non-protein component in the composition (in dry form). In some embodiments, the wt. % of the CR or a salt thereof or a dipeptide or salt thereof, or a tripeptide or salt thereof, comprising CR is at least 15 wt. % of the amino acid entities or total components in the composition (e.g., in dry form), e.g., the wt. % of the CR or a salt thereof or a dipeptide or salt thereof, or a tripeptide or salt thereof, comprising CR is at least: 20 wt. %, 25 wt. %, 30 wt. %, 35 wt. %, or more of amino acid entities or total components in the composition (e.g., in dry form), but not more than 60 wt. % of the amino acid entities or total components in the composition (e.g., in dry form).

In some embodiments, CR is present at a greater wt. % than any other amino acid entity or non-protein component in the composition (in dry form). In some embodiments, the wt. % of the CR is at least 15 wt. % of the amino acid entities or total components in the composition (in dry form), e.g., the wt. % of the CR is at least: 20 wt. %, 25 wt. %, 30 wt. %, 35 wt. %, or more of amino acid entities or total components in the composition (in dry form), but not more than 60 wt. % of the amino acid entities or total components in the composition (in dry form).

iv. Molecules to Exclude or Limit from the Composition

In some embodiments, the composition does not comprise a peptide of more than 20 amino acid residues in length (e.g., protein supplement) chosen from or derived from one, two, three, four, five, six, seven, eight, nine, 10, 11, 12, 13, 14, 15, or more (e.g., all) of egg white protein, milk protein, soy protein, casein, caseinate, hemp protein, pea protein, wheat protein, oat protein, spirulina, microprotein, lentil protein, quinoa protein, lentil protein, beef protein, or brown rice protein, or if the peptide is present, the peptide is present at less than: 10 weight (wt.) 5 wt. %, 1 wt. %, 0.1 wt. %, 0.05 wt. %, 0.01 wt. %, of the total wt. of amino acid entities or total components in the composition (e.g., in dry form).

In some embodiments, the composition comprises a combination of 3 to 19, 3 to 18, 3 to 17, 3 to 16, 3 to 15, or 3 to 10 different amino acid entities, e.g., the combination comprises at least: 42 wt. %, 75 wt. %, or 90 wt. % of the total wt. % of amino acid entities or total components in the composition (in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entities or total components in the composition (e.g., in dry form). In some embodiments, at least 50%, 60%, 70%, or more of the total grams of amino acid entities or total components in the composition (e.g., in dry form) are from one, two, three, four, or five amino acid entities of (a)-(c) or one, two, three, four, five, or six amino acid entities of (a)-(d). In some embodiments, at least: 50%, 60%, 70%, or more of the calories from amino acid entities or total components in the composition (e.g., in dry form) are from one, two, three, four, or five amino acid entities of (a)-(c) or one, two, three, four, five, or six amino acid entities of (a)-(d).

In some embodiments, lysine is absent, or if present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, trimethyllysine is absent, or if present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, carnitine is absent, or if present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form).

In some embodiments, the creatine entity does not comprise one or both of L-arginine or glycine. In some embodiments, arginine is absent, or if present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, glycine is absent, or if present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form).

In some embodiments, the ALCAR entity does not comprise one, two, or three of lysine, trimethyllysine, or carnitine. In some embodiments, the ALCAR entity does not comprise lysine, or if lysine is present, lysine is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, the ALCAR entity does not comprise carnitine, or if carnitine is present, carnitine is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, the ALCAR entity does not comprise trimethyllysine, or if trimethyllysine is present, trimethyllysine is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form).

In some embodiments, the NAC entity does not comprise one, two, three, or four of cysteine, methionine, serine, or cystine. In some embodiments, the NAC entity does not comprise cysteine, or if cysteine is present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, the NAC entity does not comprise methionine, or if methionine is present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, the NAC entity does not comprise serine, or if serine is present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form). In some embodiments, the NAC entity does not comprise cystine, or if cystine is present, is present at less than: 10 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less of the total wt. of protein components or total components of the composition (e.g., in dry form).

In some embodiments, a carbohydrate (e.g., one, two, three, four, five, six, seven, eight, nine, 10, 11, 12, 13, 14, 15, 16, 17, 18, 19, 20, 21, 22, 23, 24, 25, 26, or 27 of dextrose, maltodextrose, sucrose, dextrin, dextran, oligoosaccharide, polysaccharide, amylopectin, inulin, fructose, galactose, glucose, glycogen, high fructose corn syrup, honey, inositol, invert sugar, lactose, levulose, maltose, molasses, sugarcane, xylose, ribose, nutriose, isomalitulose, or candy) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B5, vitamin B6, vitamin B7, vitamin B9, vitamin B12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, ornithine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, molybdenum is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, a stable isotope (e.g., one, two, three, or four of isotopically labeled C, H, O, or N) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a protease (e.g., one or both of papain or bromelain) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, α-lipoic acid (ALA) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, an insulin mimetic compound (e.g., one, two, three, or four of cinnamon, vanadium (e.g., bis(picolinato)oxovanadium or bis(glycinato)oxovanadium), banaba plant extract, or corosolic acid) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, an antibacterial agent (e.g., one, two, three, or four of alanine, γ-aminobutyric acid, γ-aminolevulinic acid, or γ-aminovaleric acid) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a polymer (e.g., a cyanoacrylate polymer) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, nicotinamide riboside is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a quinone (e.g., pyrroloquinoline quinone) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of phosphatidylserine or phosphatidylcholine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, taurine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, three, four, five, or six of quercetin, astaxanthin, epigallocatechin gallate, melatonin, ginkgo biloba extract, or curcumin is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 2,4-disulfonyl α-phenyl tertiary butyl nitrone is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, three, or four of S—(N,N-diethylcarbamoyl)glutathione (carbamathione), ebselen, glutathione monoethylester, or a Szeto-Schiller peptide is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of α-tocopherol or S-adenosylmethionine (SAM) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, three, four, or five of phenylalanine, histidine, tryptophan, methionine, or tyrosine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). Without wishing to be bound by theory, one, two, three, four, or five of phenylalanine, histidine, tryptophan, methionine, or tyrosine may interfere and/or compete with the transport of BCAAs (one, two, or three of leucine, isoleucine, or valine) at the blood brain barrier.

In some embodiments, an alkaloid (e.g., one or both of huperzine A or vinpocetine) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a fatty acid (e.g., one, two, three, or four of an omega 3 fatty acid (e.g., one, two, three, four, or five of EPA, DHA, STA, DPA, or ALA), an omega 6 fatty acid (e.g., one, two, or three of GLA, DGLA, or LA), a medium chain triglyceride, or a medium chain fatty acid) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Methods of Treatment

The disclosure provides a method for improving one or both of neuronal function or cognitive function, comprising administering to a subject in need thereof an effective amount of a composition disclosed herein (e.g., an Active Moiety). The composition can be administered according to a dosage regimen described herein to improve one or both of neuronal function or cognitive function in a subject (e.g., a human).

In some embodiments, the subject does not have Alzheimer's disease. In some embodiments, the subject does not have Mild Cognitive Impairment (MCI).

The disclosure provides a method for treating or preventing neuronal injury (e.g., traumatic brain injury (TBI) or stroke), comprising administering to a subject in need thereof an effective amount of a composition disclosed herein (e.g., an Active Moiety). The composition can be administered according to a dosage regimen described herein to treat neuronal injury in a subject (e.g. a human).

In some embodiments, the composition described herein (e.g., the Active Moiety) is for use as a medicament in improving one or both of neuronal function or cognitive function in a subject (e.g., a subject with neuronal injury (e.g., TBI or stroke). In some embodiments, the composition is for use as a medicament in treating (e.g., reversing, reducing, ameliorating, or preventing) neuronal injury in a subject.

In some embodiments, the composition described herein (e.g., the Active Moiety) is for use in the manufacture of a medicament for improving one or both of neuronal function or cognitive function in a subject (e.g., a subject with neuronal injury (e.g., TBI or stroke). In some embodiments, the composition (e.g., the Active Moiety) is for use in the manufacture of a medicament for treating (e.g., reversing, reducing, ameliorating, or preventing) neuronal injury in a subject.

In some embodiments, the subject with neuronal injury (e.g., TBI or stroke) has not received prior treatment with the composition described herein (e.g., a naïve subject). In some embodiments, the subject has been diagnosed with TBI, e.g., mTBI. In some embodiments, the subject has not been diagnosed with stroke, e.g., ischemic stroke. In some embodiments, the subject is a human.

In some embodiments, the subject is at risk of or has TBI. In some embodiments, the subject is at risk of or has mild TBI. In some embodiments, the subject is at risk of or has chronic traumatic encephalopathy. In some embodiments, the subject is at risk of or has a sports-related injury. In some embodiments, the subject is at risk of or has mTBI caused by one or both of a sports-related incident or mild blunt trauma.

In some embodiments, the subject is at risk of or has stroke. In some embodiments, the subject is at risk of or has ischemic stroke. In some embodiments, the subject is at risk of or has acute ischemic stroke. In some embodiments, the subject is at risk of or has a hemorrhagic stroke. In some embodiments, the subject is at risk of or has an intracerebral hemorrhage or a subarachnoid hemorrhage. In embodiments, the subject suffers from an acute middle cerebral artery (MCA) ischemic stroke. In some embodiments, the subject has a transient ischemic attack. A transient ischemic event generally refers to a transient (e.g., short-lived) episode of neurologic dysfunction caused by loss of blood flow (e.g., in the brain, spinal cord, or retina) without acute infarction (e.g., tissue death).

In embodiments, the subject has a first-time stroke (e.g., the subject has no prior history of stroke). In embodiments, the subject has previously had a stroke (e.g., the subject has had one, two, three, four, or more prior events of stroke). In some embodiments, a subject exhibits a symptom of neuronal injury (e.g. TBI or stroke), e.g., a physical, mental health, neuronal, or physiological symptom of neuronal injury, prior to administration of the composition.

In some embodiments, a subject exhibits a mental health or physical symptom of TBI chosen from one, two, three, four, five, six, seven, eight, nine, 10, 11, 12, 13, 14, 15, 16, 17, 18, or more (e.g., all) of cognitive deficits, dizziness, hearing loss, headache (e.g., frequent headache), loss of consciousness, memory loss, confusion, sleep disturbance, nausea, decreased balance, fatigue, drowsiness, blurred vision, ringing in ears, sensitivity to light, sensitivity to sound, decreased ability to concentration, mood swings, or increased anxiety (e.g., relative to a healthy subject without TBI).

In some embodiments, the subject exhibits a mental or physical symptom of stroke (e.g., ischemic stroke) chosen from one, two, three, four, five, six, seven, eight, nine, 10, 11, 12, 13, 14, 15, 16, or more (e.g., all) of: numbness, decreased balance, memory loss, facial weakness, eyelid dropping, paralysis (e.g., hemiplegia), decreased sensory sensation, decreased reflexes, tongue weakness, involuntary eye moment, a visual field defect, aphasia, increased confusion, vertigo, decreased speaking ability (e.g., apraxia), decreased walking ability, or decreased motor coordination (e.g., relative to a healthy subject without stroke).

In some embodiments, the subject exhibits a physiological symptom of stroke chosen from one, two, three, four, five, six, seven, eight, nine, 10, 11, 12, or more (e.g., all) of thrombotic activation, cerebral hypoperfusion, hypoxia, anaerobic glycolysis, ATP depletion, one or both of Na/K ATPase dysfunction or depolarization, excitotoxicity, NMDA-AMPA receptor activation, mitochondrial injury, apoptosis, increased calcium influx, increased free radicals, or oxidative stress.

In some embodiments, the method further includes monitoring a subject for an improvement in one or more symptoms of neuronal injury (e.g. TBI or stroke) described herein, e.g., a physical, mental health, neuronal, or physiological symptom of neuronal injury, prior to administration of the composition. In some embodiments, administration of the composition (e.g., at a dosage regimen described herein) results in an improvement in one or more symptoms of neuronal injury (e.g. TBI or stroke) described herein.

In some embodiments, administration of the composition (e.g., at a dosage regimen described herein) results in an improvement in a mental health or physical symptom of TBI chosen from one, two, three, four, five, six, seven, eight, nine, 10, 11, 12, 13, 14, 15, 16, 17, 18, or more (e.g., all) of cognitive deficits, dizziness, hearing loss, headache (e.g., frequent headache), loss of consciousness, memory loss, confusion, sleep disturbance, nausea, decreased balance, fatigue, drowsiness, blurred vision, ringing in ears, sensitivity to light, sensitivity to sound, decreased ability to concentration, mood swings, or increased anxiety (e.g., relative to the subject prior to administration of the composition).

In some embodiments, administration of the composition (e.g., at a dosage regimen described herein) results in an improvement in a mental health or physical symptom of stroke (e.g., ischemic stroke) chosen from one, two, three, four, five, six, seven, eight, nine, 10, 11, 12, 13, 14, 15, 16, or more (e.g., all) of: numbness, decreased balance, memory loss, facial weakness, eyelid dropping, paralysis (e.g., hemiplegia), decreased sensory sensation, decreased reflexes, tongue weakness, involuntary eye moment, a visual field defect, aphasia, increased confusion, vertigo, decreased speaking ability (e.g., apraxia), decreased walking ability, or decreased motor coordination (e.g., relative to a healthy subject without stroke).

In some embodiments, administration of the composition (e.g., at a dosage regimen described herein) results in an improvement in a mental health or physical symptom of transient ischemic attack chosen from one, two, three, four, five, six, seven, eight, nine, or more (e.g., all) of: motor weakness (e.g., one-sided motor weakness, e.g., one-sided facial droop), numbness (e.g., one-sided numbness), paralysis (e.g., one-sided paralysis), double vision, amaurosis fugax, vertigo, decreased balance, blindness, headache, or difficulty speaking.

In some embodiments, administration of the composition (e.g., at a dosage regimen described herein) results in an improvement in a physiological symptom of stroke choesn from one, two, three, four, five, six, seven, eight, nine, 10, 11, 12, or more (e.g., all) of thrombotic activation, cerebral hypoperfusion, hypoxia, anaerobic glycolysis, ATP depletion, one or both of Na/K ATPase dysfunction or depolarization, excitotoxicity, NMDA-AMPA receptor activation, mitochondrial injury, apoptosis, increased calcium influx, increased free radicals, or oxidative stress.

In some embodiments, administration of the composition (e.g., at a dosage regimen described herein) to a subject with neuronal injury (e.g., TBI or stroke) results in one, two, three, four, five, six, seven, eight, nine, or all of: a) increased TCA cycle anaplerosis or ATP production; b) decreased mitochondrial calcium influx; c) decreased free radicals or reactive oxidative species (ROS); d) decreased pro-inflammatory cytokines, e.g., from the activation of one or both of abnormal microglia or astrocyte; e) decreased microglial response to pro-inflammatory signals (e.g., M1 microglia phenotype to M2 microglia phenotype); f) increased neuronal signaling (e.g. hippocampal signaling); g) decreased inflammation (e.g., inflammation of brain tissue); h) increased ionic flux; i) increased mitochondrial function; or j) decreased synaptic dysfunction, comprising administering to a subject in need thereof an effective amount of a composition (e.g., an Active Moiety) of any of the aspects or embodiments disclosed herein, thereby improving one, two, three, four, five, six, seven, eight, nine, or all of (a)-(j).

In some embodiments, administration of a composition described herein results in enhanced recovery in a subject with TBI (e.g., mild TBI or severe forms of TBI) or stroke. In some embodiments, administration of the composition results in improved survival of a subject with TBI (e.g., mild TBI or severe forms of TBI) or stroke.

In some embodiments, administration of a composition described herein improves symptoms of neuronal injury (e.g., TBI or stroke), e.g., by reducing inflammation. In some embodiments, administration of a composition increases one or both of GSH synthesis or OS scavenging, thereby decreasing inflammation in a patient with TBI.

In some embodiments, administration of the composition described herein (e.g., the Active Moiety) has neuroprotective effects, e.g., in a subject during or after sports-related activities.

In some embodiments, onset time of neuronal injury (e.g., TBI or stroke) is determined by a medical professional, e.g., by questioning regarding one or more symptoms of neuronal injury as described herein. In some embodiments, brain imaging (e.g., MRI) is used to determine one or both of the time of onset or duration of neuronal injury (e.g., TBI or stroke) in a subject. In some embodiments, the composition is administered following neuronal injury, e.g., within 30 minutes, 1 hour, 2 hours (h), 3 h, 4 h, 5 h, 6 h, 8 h, 12 h, 16 h, 18 h, 20 h, 24 h, 48 h, or 72 h after the onset time of neuronal injury.

A composition described herein can be administered to a subject prior to or following a head injury (e.g., a concussion). In some embodiments, the composition is administered prior to a head injury, e.g., 30 minutes, 1 hour, 2 h, 3 h, 4 h, 5 h, 6 h, 8 h, 12 h, 16 h, 18 h, 20 h, 24 h, 48 h, or 72 h prior to a head injury (e.g., a concussion). In some embodiments, the composition is administered following a head injury, e.g., within 30 minutes, 1 hour, 2 h, 3 h, 4 h, 5 h, 6 h, 8 h, 12 h, 16 h, 18 h, 20 h, 24 h, 48 h, or 72 h after a head injury (e.g., a concussion). In some embodiments, the composition is administered both prior to and following a head injury (e.g., a concussion).

In some embodiments, levels (e.g., levels in plasma) of BCAAs are decreased in a subject with neuronal injury (e.g., TBI or stroke) prior to treatment with composition described herein, e.g., relative to a healthy subject without neuronal injury. In some embodiments, levels (e.g., plasma levels) of one, two, or three of valine, leucine, or isoleucine are decreased in a subject with neuronal injury prior to treatment with the composition. In an embodiment, levels of valine are decreased in a subject with neuronal injury prior to treatment with the composition, e.g., the decreased levels of valine are associated with mortality in a subject with neuronal injury (e.g., TBI).

In some embodiments, administration of the composition described herein can balance the profile of amino acids in a subject with neuronal injury (e.g., TBI, e.g., mTBI, or stroke). In some embodiments, administration of the composition increases the level (e.g., plasma level) of one, two, or more (e.g., all) of valine, leucine, or isoleucine in a subject with neuronal injury, e.g., relative to the subject prior to treatment with the composition. In an embodiment, administration of the composition increases the level (e.g., plasma level) of valine in a subject with neuronal injury (e.g., TBI), e.g., relative to the subject prior to treatment with the composition.

In some embodiments, the method further includes performing one, two, three, or four of a Self-Assessment of Cognition (SAC), a Sport Concussion Assessment Tool 5th Edition (SCAT5) assessment, Disability Rating Scale (DRS), or a Balance Error Scoring System (BESS) assessment of the subject with neuronal injury, e.g., prior to or after administration of the composition described herein. In some embodiments, administration of the composition results in an improvement in one, two, three, or four of a SAC, SCAT5, DRS, or BESS score of the subject.

In some embodiments, the method can further include performing a Disability Rating Scale (DRS) to assess improvements in a subject with TBI. In some embodiments, administration of a composition including amino acid entities results in a decrease in the DRS score of the patient. The DRS is described in Neese et al., Brain Inj. 2000 August; 14(8):719-24, which is hereby incorporated by reference in its entirety.

In some embodiments, the method can further include performing a Self-Assessment of Cognition (SAC) to assess improvements in a subject with TBI. In some embodiments, administration of a composition including amino acid entities results in an increase in the SAC score of the patient. The SAC is described in Neese et al., Brain Inj. 2000 August; 14(8):719-24, which is hereby incorporated by reference in its entirety.

In some embodiments, the method can further include performing a Balance Error Scoring System (BESS) to assess improvements in a subject with TBI. In some embodiments, administration of a composition including amino acid entities results in a decrease in the BESS score of the patient. The BESS is described in Bell et al., Sports Health. 2011 May; 3(3): 287-295, which is hereby incorporated by reference in its entirety.

### Dosage Regimens

The composition (e.g., the Active Moiety) can be administered according to a dosage regimen described herein to improve one or both of neuronal function or cognitive function in a subject, e.g., treat or prevent neuronal injury (e.g., traumatic brain injury (TBI) or stroke) in a subject in need thereof (e.g, a human).

In some embodiments, the composition can be provided to a subject with neuronal injury (e.g., TBI or stroke) in either a single or multiple dosage regimen. In some embodiments, a dose is administered twice daily, three times daily, four times daily, five times daily, six times daily, seven times daily, or more. In certain embodiments, the composition is administered one, two, or three times daily. In some embodiments, the composition is administered for at least 2 days, 3 days, 4 days, 5 days, 6 days, 7 days, or 2 weeks. In some embodiments, the composition is administered chronically (e.g., more than 30 days, e.g., 31 days, 40 days, 50 days, 60 days, 3 months, 6 months, 9 months, one year, two years, or three years).

The composition can be administered every 2 hours, every 3 hours, every 4 hours, every 5 hours, every 6 hours, every 7 hours, every 8 hours, every 9 hours, or every 10 hours to one or both of neuronal function or cognitive function in a subject (e.g., a subject with neuronal injury, e.g., TBI or stroke).

In some embodiments, the composition is administered prior to a meal (e.g., breakfast, lunch or dinner). In other embodiments, the composition is administered concurrent with a meal (e.g., breakfast, lunch or dinner). In other embodiments, the composition is administered following a meal (e.g., breakfast, lunch or dinner). In certain embodiments, the composition is administered after breakfast and dinner.

In some embodiments, the composition comprises three stick packs, e.g., each stick pack comprising 33.3%+/−15% of the quantity of each amino acid entity included in the composition described herein. In certain embodiments, three stick packs are administered two times daily.

In some embodiments, the composition comprises four stick packs, e.g., each stick pack comprising 25%+/−15% of the quantity of each amino acid entity included in the composition described herein. In certain embodiments, four stick packs are administered two times daily.

In some embodiments, the composition is administered at a dose of 5 grams (g)+/−20% to 150 g+/−20% total amino acid entities daily, e.g., 15 g+/−20% to 100 g+/−20% total amino acid entities daily, e.g., in one dose per day, two doses per day, three doses per day, four doses per day, in five doses per day, six doses per day, seven doses per day, eight doses per day, nine doses per day, or ten doses per day (e.g., one dose or two doses per day).

In some embodiments, the composition is administered at a dosage of 15 g+/−20% to 100 g+/−20% total amino acid entities daily. In som embodiments the composition is administered at a dosage of: 20 g+/−20%, 25 g+/−20%, 30 g+/−20%, 33 g+/−20%, 34 g+/−20%, 35 g+/−20%, 40 g+/−20%, 45 g+/−20%, 50 g+/−20%, 53 g+/−20%, 54 g+/−20%, 55 g+/−20%, 60 g+/−20%, 65 g+/−20%, 70 g+/−20%, 75 g+/−20%, 80 g+/−20%, or 85 g+/−20% total amino acid entities daily.

In some embodiments, the composition is administered at a dosage of 2 g+/−20% to 60 g+/−20% total amino acid entities, e.g., once daily, twice daily, three times daily, four times daily, five times daily, or six times daily (e.g., twice daily). In some embodiments, the composition is administered at a dose of 2 g+/−10% to 10 g+/−20%, 10 g+/−20% to 20 g+/−20%, 20 g+/−20% to 40 g+/−20%, or 40 g+/−20% to 60 g+/−20% total amino acid entities, e.g., once daily, twice daily, or three times daily (e.g., twice per day). In certain embodiments, the composition is administered at a dose of 10 g+/−20% to 20 g+/−20% total amino acid entities twice daily, e.g., 17 g+/−20% total amino acid entities twice daily. In certain embodiments, the composition is administered at a dose of 20 g+/−20% to 40 g+/−20% total amino acid entities twice daily, e.g., 27 g+/−20% total amino acid entities twice daily.

In some embodiments, the composition is administered at a dosage of 10 g+/−20%, 15 g+/−20%, 16+/−20%, 17+/−20%, 18+/−20%, 19+/−20%, 20 g+/−20%, 21 g+/−20%, 22 g+/−20%, 23 g+/−20%, 24 g+/−20%, 25 g+/−20%, 26 g+/−20%, 27 g+/−20%, 28 g+/−20%, 29 g+/−20%, 30 g+/−20%, 35 g+/−20%, 40 g+/−20%, 45 g+/−20%, or 50 g+/−20%, e.g., two times daily. In certain embodiments, the composition is administered at a dosage of 17 g+/−20% two times daily. In certain embodiments, the composition is administered at a dosage of 27 g+/−20% two times daily.

### Production of Active Moiety and Pharmaceutical Compositions

The present disclosure features a method of manufacturing or making a composition (e.g., an Active Moiety) of the foregoing invention. Amino acid entities used to make the compositions may be agglomerated, and/or instantized to aid in dispersal and/or solubilization.

The compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the compositions of the instant disclosure, the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.1, 0.01, or 0.001% (w/w)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include microbes, endotoxins, metals, or a combination thereof. In some embodiments, the level of contamination, e.g., by metals, lecithin, choline, endotoxin, microbes, or other contaminants (e.g., contaminants from raw materials) of each portion of a composition is below the level permitted in food.

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g. Alcolec F100), sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard #4306, Nat Orange WONF #1326, lime 865.0032U, and lemon 862.2169U), a bitterness masking agent (e.g. 936.2160U), and natural or artificial colorings (e.g. FD&C Yellow 6). Exemplary ingredient contents for each stick pack are shown in Table 4.

TABLE 4
Ingredient contents in each stick pack.

| INGREDIENT | GRADE | FUNCTION | SOURCE; COMMENT |
|---|---|---|---|
| Ammo Acids | USP | Active Pharmaceutical Ingredient | Various sources; Non- |
| | | (API) | instantized form (MFG |
| | | | scale) |
| Citric Acid | USP | pH, Flavor | Spectrum Chems; |
| | | | f(volume) ≤1.0% w/v |
| Acesulfame K | NF | Sweetness (rapid onset) | Spectrum Chems; Target 1 |
| | | | Sweetener |
| Sucralose | NF | Sweetness (slow onset) | Spectrum Chems; WHO |
| | | | ADI ≤15 mg/kg |
| Lecithin (Alecolec F100) | FCC | Wetting Agent | American Lecithin |
| | | | Company |
| Xanthan Gum | FCC | Stabilizer/Thickener | TIC Gums; f(volume) ≤0.5% |
| | | | w/v |
| Vanilla Custard (Art) | GRAS | Taste/Aroma | David Michael; Mask |
| | | | sulfur |
| Orange (Natural and | GRAS | 1° flavor | David Michael; Citrus |
| WONF) | | | profile matches low pH |
| Lime (Natural and | GRAS | 2° flavor | FONA; Single flavor |
| WONF) | | | supplier |
| Lemon (Natural and | GRAS | 2° flavor | FONA; Single flavor |
| artificial) | | | supplier |
| Taste Modifier | GRAS | Bitterness masking | FONA; Useful at low |
| | | | volume |
| FD&C Yellow No. 6 | USP | Color | Sensient; Match flavor |
| | | | profile |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 5).

TABLE 5
Exemplary contents in each stick pack.

| INGREDIENT | GRADE | FUNCTION |
|---|---|---|
| Amino Acids | USP | Active Pharmaceutical |
| | | Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat + WONF) | GRAS | 1° flavor |
| FD&C Yellow No. 6 | USP | Color |

### Dietary Compositions

The composition (e.g., Active Moiety) including amino acid entities can be formulated and used as a dietary composition, e.g., chosen from a medical food, a functional food, or a supplement. In such an embodiment, the raw materials and final product should meet the standards of a food product.

The composition of any of the aspects and embodiments disclosed herein can be for use as a dietary composition, e.g., chosen from a medical food, a functional food, or a supplement. In some embodiments, the dietary composition is for use in a method, comprising administering the composition to a subject. The composition can be for use in a dietary composition for the purpose of improving one or both of neuronal function or cognitive function in a subject.

In some embodiments, the dietary composition is chosen from a medical food, a functional food, or a supplement. In some embodiments, the composition is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition described herein for use in the management of neuronal injury (e.g., traumatic brain injury (TBI) or stroke) in a subject.

The present disclosure features a method of of improving one, two, three, four, five, six, seven, eight, nine, or all of: a) decreased TCA cycle anaplerosis or ATP production; b) increased mitochondrial calcium influx; c) increased free radicals or reactive oxidative species (ROS); d) increased pro-inflammatory cytokines, e.g., from the activation of one or both of abnormal microglia or astrocyte; e) increased microglial response to pro-inflammatory signals (e.g., M1 microglia phenotype to M2 microglia phenotype); f) decreased neuronal signaling (e.g. hippocampal signaling); g) increased inflammation (e.g., inflammation of brain tissue); h) decreased ionic flux; i) decreased mitochondrial function; or j) decreased synaptic dysfunction, comprising administering to a subject an effective amount of a composition (e.g., a dietary composition) described herein.

The present disclosure features a method of providing nutritional support or supplementation to a subject with neuronal injury (e.g., TBI or stroke), comprising administering to the subject an effective amount a composition (e.g., a dietary composition) described herein.

The present disclosure features a method of providing nutritional support or supplementation that aids in the management of neuronal injury (e.g., TBI or stroke), comprising administering to a subject in need thereof an effective amount of a composition (e.g., a dietary composition) described herein.

In some embodiments, the subject is at risk of or has TBI. In some embodiments, the subject is at risk of or has mild TBI. In some embodiments, the subject is at risk of or has chronic traumatic encephalopathy. In some embodiments, the subject is at risk of or has a sports-related injury. In some embodiments, the subject is at risk of or has mTBI caused by one or both of a sports-related incident or mild blunt trauma.

In some embodiments, the subject is at risk of or has stroke. In some embodiments, the subject is at risk of or has ischemic stroke. In some embodiments, the subject is at risk of or has acute ischemic stroke. In some embodiments, the subject is at risk of or has a hemorrhagic stroke. In some embodiments, the subject is at risk of or has an intracerebral hemorrhage or a subarachnoid hemorrhage. In embodiments, the subject suffers from an acute middle cerebral artery (MCA) ischemic stroke. In embodiments, the subject has a first-time stroke (e.g., the subject has no prior history of stroke). In embodiments, the subject has previously had a stroke (e.g., the subject has had one, two, three, four, or more prior events of stroke).

The compositions can be used in methods of dietary management of a subject (e.g., a subject without neuronal injury (e.g., TBI or stroke). In some embodiments, the subject does not have neuronal injury (e.g., TBI or stroke).

In some embodiments, the subject is at risk of or has a sports-related injury.

### Biomarkers

Any of the methods disclosed herein can include evaluating or monitoring the effectiveness of administering a composition of the invention as described herein (e.g., the Active Moiety) to a subject with neuronal injury (e.g., traumatic brain injury (TBI) or stroke). The method includes acquiring a value of effectiveness to the composition, such that the value is indicative of the effectiveness of the therapy.

In embodiments, the value of effectiveness to the composition in treating a subject with neuronal injury (e.g., TBI or stroke) comprises a measure of one, two, three, four, five, or more (e.g., all) of the following: a) a level of glutathione (GSH); b) level of neurofilament light (NF-L); c) a level of phosphorylated tau (p-tau); d) a level of total tau; e) level of ubiquitin C-terminal hydrolase 1 (UCHL-1); or f) a level of glial fibrillary acidic protein (GFAP).

In some embodiments of any of the methods disclosed herein, the measure of one or more of a)-f) is obtained from a sample acquired from the subject with neuronal injury (e.g., TBI or stroke). In some embodiments, the sample is chosen from a blood sample (e.g., a plasma sample) or a tissue sample (e.g., a brain tissue sample).

In some embodiments, the level of one, two, three, four, five, or more (e.g., all) of a)-f) are assessed in a blood sample (e.g., a plasma sample) from a subject with neuronal injury (e.g., TBI or stroke). In some embodiments, the subject has a sports-related injury.

In some embodiments, the subject is evaluated prior to receiving, during, or after receiving, a composition described herein.

In some embodiments, administration of the composition including amino acid entities (e.g., at a dosage regimen described herein), results in one, two, three, four, five, or more (e.g., all) of the following: a) increased level of GSH; b) decreased level of NF-L; c) decreased level of p-tau; d) decreased level of total tau; e) decreased level of UCHL-1; or f) decreased level of GFAP.

### EXAMPLES

The Examples below are set forth to aid in the understanding of the inventions, but are not intended to, and should not be construed to, limit its scope in any way.

### Example 1. Cytokine Levels in Microglial Cells Treated with Amino Acid Compositions

Neuroinflammation and microglial activation are key mediators of repair and recovery after traumatic brain injury (TBI). However, recent clinical and laboratory data have shown that TBI can cause persistent neuroinflammation and microglial activation, in some cases lasting many years, and can lead to chronic neurodegeneration, dementia and encephalopathy. Prospective studies of TBI biomarkers in adults with severe TBI have shown that serum levels of IL-1β, IL-6, CXCL8, IL-10, and tumour necrosis factor (TNFα) are chronically increased.

Pups were decapitated, brains removed from the skulls, cortices dissected and collected in Calcium and Magnesium free Hanks Balanced Salt Solution (CMF-HBSS) on ice. Cortices were chopped with a sterile razor blade in chop solution (CMF-HBSS) and digested in 2 ml digestion solution (0.25% trypsin in CMF-HBSS) in a 15 ml sterile plastic tube for 20 minutes at 37° C. in a water bath. After removing as much as possible of the digestion solution (approximately 1 ml), the digestion was stopped by adding 3 ml stop solution (0.01% DNaseI and 0.5% trypsin inhibitor in FCS). Cortices were triturated 10-15 times with a fire-polished Pasteur pipette and centrifuged at 300 g for 5 minutes. The pellet was resuspended in 1 ml nutrition medium (10% FCS, 1% Penicillin-Streptomycin, 2 mM L-glutamine in DMEM) per pup. Cells were seeded into poly-D-lysine pre-coated T75 flasks containing 14 ml nutrition medium (one pup per flask). Medium was changed every 3 to 4 days and cells were cultured at 37° C., 95% humidity and 5% CO2 for approximately 14 days before harvesting the microglia.

After approximately 14 days of cultivation microglia cells were harvested by shaking the T75 flasks at 200 rpm at 37° C. for 2 h. After visual inspection confirming that the microglia cells had detached, the supernatants containing the microglia were transferred to 50 ml tubes (supernatant from 1 flask into 1 tube) and centrifuged at 300 g for 10 minutes. The pelleted microglia cells were resuspended in 2 ml of nutrition medium. Microglia cells were seeded into uncoated plates 1×105 cells/well (96 well plate) and incubated for 2 h at 37° C. Then the plates were rocked at 100 rpm for 5 minutes to detach the oligodendrocytes. The medium containing the oligodendrocytes was exchanged with fresh medium and microglia cells were incubated for 2 days at 37° C. before LPS treatment. 24 h before LPS stimulation single amino acids and combinations were administered. Next, 1 h before LPS stimulation, medium was changed to treatment medium (DMEM, 5% FBS, 2 mM L-glutamine) containing amino acids or combinations. Cells were maintained in treatment medium for the remaining culture period. One hour after changing cells to treatment medium, cells were stimulated with LPS (100 ng/ml) for 12 h in total. Cells treated with vehicle, LPS alone served as controls.

After 12 h of LPS stimulation, cell supernatants were collected and cytokines IL-6 and TNFα were measured.

The experiment was performed in six technical replicates for all groups.

Cytokine Measurements

Levels of TNFα an IL-6 were measured in supernatants of primary microglia cells collected 12 h after LPS stimulation. Cytokines were measured by an immunosorbent assay (U-PLEX Custom Mouse Cytokine, Mesoscale Discovery) according to the instructions of the manufacturer and evaluated in comparison to calibration curves provided in the kit. Results are given as pg per ml.

Results:

Table 6 shows the IL-6 protein levels in mouse microglia cells activated with LPS and treated with amino acid combinations compared to vehicle treated with LPS stimulus. IL-6 is reduced by L (L-leucine), I (L-isoleucine), V (L-valine), N-acetylcysteine (NAC), acetyl-1-carnitine (ALCAR)), and creatine (CR); LIV/NAC/ALCAR; LIV/NAC; and NAC. LIV/NAC potentiates the effect of LIV and NAC on IL-6. LIV/NAC/ALCAR/CR and LIV/NAC/ALCAR potentiates the effect of single components on IL-6.

TABLE 6
IL-6 protein levels in mouse microglia cells activated with LPS and treated with
amino acid combinations.

| | | IL-6 Measurements | | | | | | | |
|---|---|---|---|---|---|---|---|---|---|
| | | | | Number | P- | | | | P-value |
| Amino Acid | | | Std. | of | value | P-value | P-value | P-value | to |
| Supplement | Conc. (X) | Mean | Deviation | replicates | to LPS | to LIV | to NAC | to CR | ALCAR |
| LIV/NAC/ | 2.5 mM/1 mM/1 mM/ | 37198 | 10852 | 6 | 0.0013 | 0.0449 | 0.1075 | 0.0038 | 0.0050 |
| ALCAR/CR | 1 mM | | | | | | | | |
| LIV/NAC/ | 2.5 mM/1 mM/1 mM | 39052 | 8236 | 6 | 0.0009 | 0.0498 | 0.1229 | | 0.0041 |
| ALCAR | | | | | | | | | |
| LIV/NAC | 2.5 mM/1 mM | 35100 | 6097 | 6 | 0.0002 | 0.0113 | 0.0149 | | |
| LIV | 2.5 mM | 52671 | 12486 | 6 | 0.0894 | | | | |
| NAC | 1 mM | 46749 | 7574 | 6 | 0.0069 | | | | |
| CR | 1 mM | 56492 | 6435 | 6 | 0.1170 | | | | |
| ALCAR | 1 mM | 59600 | 10803 | 6 | 0.3688 | | | | |

Table 7 shows the TNFα protein levels in mouse microglia cells activated with LPS and treated with amino acid combinations compared to vehicle treated with LPS stimulus. TNFα is reduced by LIV/NAC/ALCAR/CR, LIV/NAC/ALCAR, LIV/NAC and NAC. LIV/NAC potentiates the effect of LIV and NAC on TNFα. LIV/NAC/ALCAR/CR and LIV/NAC/ALCAR potentiates the effect of single components on TNFα.

TABLE 7
TNFα protein levels in mouse microglia cells activated with LPS and treated with
amino acid combinations.

| | | TNFα Measurements | | | | | | | |
|---|---|---|---|---|---|---|---|---|---|
| Amino | | | | Number | | | | | P-value |
| Acid | | | Std. | of | P-value | P-value | P-value | P-value | to |
| Supplement | Conc. (X) | Mean | Deviation | replicates | to LPS | to LIV | to NAC | to CR | ALCAR |
| LIV/NAC/ | 2.5 mM/1 mM/ | 8997 | 1791 | 6 | <0.0001 | 0.0097 | 0.0010 | <0.0001 | <0.0001 |
| ALCAR/CR | 1 mM/1 mM | | | | | | | | |
| LIV/NAC/ | 2.5 mM/1 mM/ | 9992 | 1031 | 6 | <0.0001 | 0.0209 | 0.0016 | | <0.0001 |
| ALCAR | 1 mM | | | | | | | | |
| LIV/NAC | 2.5 mM/1 mM | 10752 | 1175 | 6 | <0.0001 | 0.0607 | 0.0055 | | |
| LIV | 2.5 mM | 13513 | 2977 | 6 | 0.0089 | | | | |
| NAC | 1 mM | 14814 | 2564 | 6 | 0.0283 | | | | |
| CR | 1 mM | 17884 | 1901 | 6 | 0.9903 | | | | |
| LCAR | 1 mM | 21897 | 3334 | 6 | 0.0226 | | | | |

### Example 2. Evaluation of the Efficacy of Amino Acid Compositions in a Rat Model of TBI

Adult male, Sprague-Dawley rats (250-350 g) from Charles River were used. Animals were housed 2 per cage in polycarbonate rat cages equipped with microisolators and acclimated for up to 7 days. All rats were examined, handled, and weighed prior to initiation of the study to assure adequate health and suitability. During the course of the study, 12/12 light/dark cycles were maintained. The room temperature was maintained between 20 and 23° C. with a relative humidity maintained around 50%. Chow and water were provided ad libitum for the duration of the study. Animals were randomly assigned across treatment groups.

Among several TBI animal models, the Controlled Cortical Impact (CCI) model is the most used and the only one commercially available. Compared to other models, the CCI model can be controlled in all mechanical factors such as time, velocity and depth of the impact, moreover, it is highly reproducible and has low mortality rate. The CCI model shares all the major pathological features that are seen in human TBI such as concussion, contusion, traumatic axonal injury, hemorrhage and neuroinflammation (Xiong Y. et al., 2013 Nature Reviews Neuroscience). Controlled cortical impact (CCI) to the parasagittal cortex was produced by an electronic cortical contusion device (Custom Design & Fabrication, Inc [CDF], Richmond, Va.). This device reliably creates a contusion injury to the exposed area of the brain with a brass-tipped impounder (Scheff et al., 1997). Rats were anesthetized with isoflurane (5%) and O2 (300 cm3/min), and mounted in a stereotaxic frame. Under aseptic conditions, a sagittal incision was made in the scalp and the fascia retracted to expose the cranium. Then, a 6-mm diameter trephine drill was used to open the skull centered approximately 4 mm lateral to the sagittal suture, mid-way between bregma and lambda. CCI brain injuries were produced with a 5-mm-diameter rounded brass impactor attached to a computer-controlled piston propelled electronically with following parameters: velocity=2.5 m/s; depth=3 mm; duration=100 ms). After CCI, any cortical surface hemorrhaging was controlled and the fascia and scalp sutured. The animals were allowed to recover in a warmed recovery chamber and appropriate post-operative care was taken.

On the day of surgery, Day 0, animals were dosed immediately after TBI within 30 mins of surgery. Treatment was subsequently delivered orally twice daily for 14 consecutive days. Dose volume was 10 mL/kg.

Fifteen rats were used in each of the following groups:
1. Sham vehicle
2. TBI vehicle
3. TBI Amino Acid Composition 1 (L-leucine, L-isoleucine, L-valine, N-acetylcysteine (NAC), and acetyl-1-carnitine (ALCAR)) Low
4. TBI Amino Acid Composition 1 High
5. TBI Amino Acid Composition 2 (L-leucine, L-isoleucine, L-valine, NAC, ALCAR, and Creatine (CR)) Low
6. TBI Amino Acid Composition 2 High

The beam balance test examined vestibulomotor reflex activity of the animals after injury. Animals were trained prior to surgery to maintain their balance on the beam for 60 sec. The rats were tested during the first week after injury at days 3, 5, and 7. Each animal was gently placed on a suspended narrow beam 1.5 cm wide with a rough surface. The duration that it remained on the beam was measured with a maximum cutoff time of 60 sec. Each animal received three trials per test day and the trials where averaged to obtain a beam balance time. Cushioned pads were placed under the beam to prevent injury in the event the animal fell.

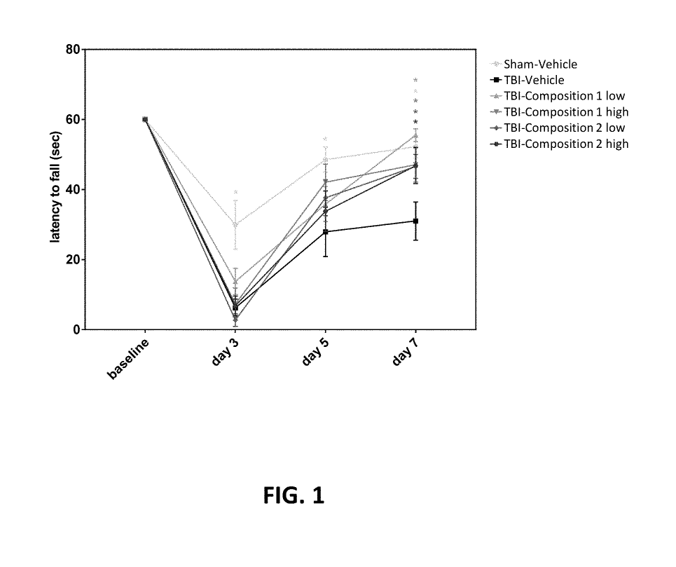

The effects of TBI treatment with the amino acid compositions on the beam balance following injury are shown in FIG. 1. Prior to injury, all animals were able to maintain their balance on the beam for at least 60 seconds. At Day 3 and Day 5, sham animals were able to maintain their balance for significantly longer than vehicle-treated controls. At Day 7 following injury, animals treated with Amino Acid Composition 1 low and high and Amino Acid Composition 2 low and high all exhibited significantly improved balance times compared to vehicle-treated controls.

The NeuroCube® Platform uses computer vision to detect changes in gait geometry and gait dynamics in rodent models of neurological disorders, pain and neuropathies. This platform is completely automated and captures both gait geometry and gait dynamics (stance, swing, propulsion, etc.).

Rats were placed in the NeuroCube® Platform for a 5 min test. The most dominant of the features collected that define the disease phenotype (symptom descriptors) were identified and ranked. Complex bioinformatic algorithms were used to calculate the discrimination probability between the Sham and the CCI rats and to detect the test compound's ability to reverse the disease phenotype.

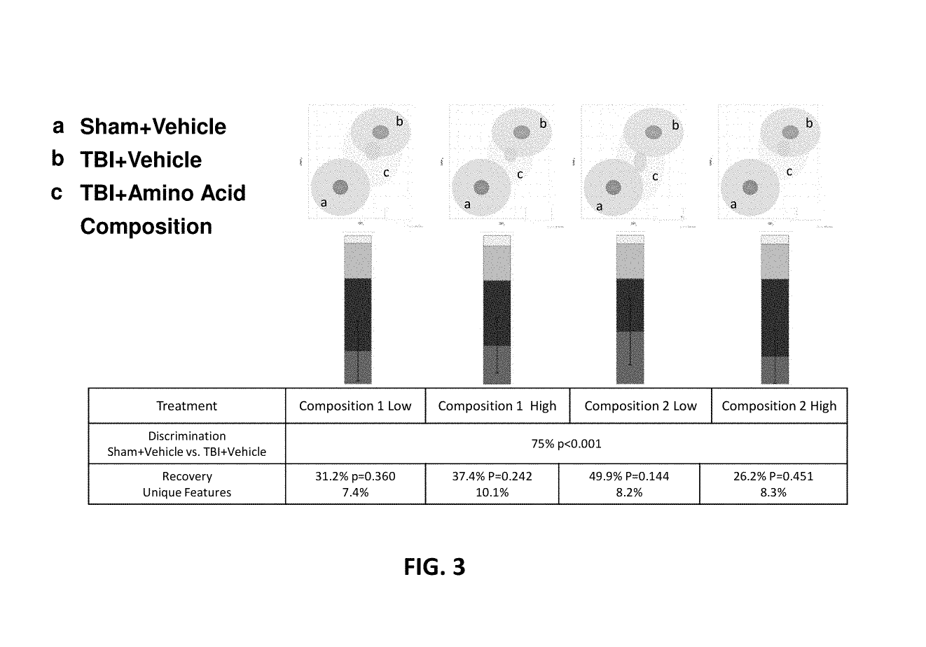

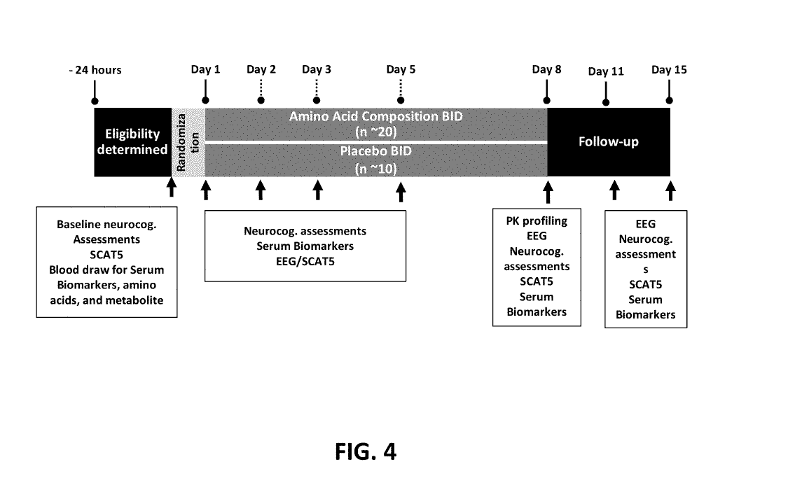

FIGS. 3 and 4 provide a graphic representation of the features and show the extent to which the test compound normalizes each of the features, including an overall percent recovery based on the effect of the compound on weighted features (weighted according to their contribution to defining the model). Rats were tested on day 10 after injury.

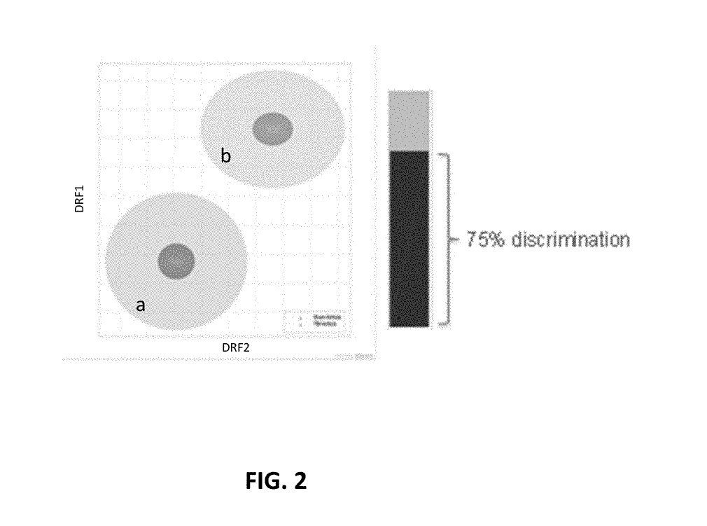

The results of NeuroCube® testing are shown in FIGS. 3 and 4. NeuroCube testing at Day 10 showed at 75% discrimination between Sham surgical controls and the TBI+Vehicle group (FIG. 2). Some evidence of recovery over all features was detected with treatment with the amino acid compositions (FIG. 3). The highest percent recovery over all features was observed with Amino Acid Composition 2 Low dose (50%, p=0.144) and Amino Acid Composition 1 High dose (37% p=0.242). NeuroCube® detected that treatment with a Amino Acid Composition 2 Low dose produced close to 50% recovery over all features. The appearance of new features—side-effects not in the direction of recovery towards the Sham controls—was limited to 7-10% in all Amino Acid Composition treatment groups.

### Example 3. Treatment of Subjects with TBI with an Amino Acid Composition

The study described herein features the administration of a composition including amino acids to patients with TBI. Adult subjects aged 18-35 years (inclusive) with mild traumatic brain injury caused by any sports related incident or blunt trauma to the head will be administered the amino acid composition. The composition includes L-leucine, L-isoleucine, L-valine, N-acetylcysteine (NAC), and acetyl-1-carnitine (ALCAR) (Table 8). Subjects will receive about 17.1 g of the amino acid composition twice daily for a total of 34.2 g daily.

TABLE 8
Exemplary components and amount of the amino acid
composition for the treatment of subjects with TBI.

| | Dry Weight (g)/ | Dry Weight (%)/ |
|---|---|---|
| Components: | Stick Pack: | Stick Pack: |
| L-Leucine | 1.6667 | 24.17 |
| L-Isoleucine | 1.6667 | 24.17 |
| L-Valine | 1.6667 | 24.17 |
| N-Acetyl-Cysteine | 0.5000 | 7.25 |
| Acetyl-L-Carnitine HCL | 0.2359 | 3.42 |
| Excipients | 1.1957 | 16.82 |
| Active Substances | 5.7000 | 83.18 |
| (excluding HCL): | | |
| Total: | 6.8957 | 100.00 |

This study is primarily intended to assess the safety, tolerability, and the pharmacokinetics of a composition administered over 8 days in adult subjects with mild traumatic brain injury (mTBI). Safety and tolerability of the amino acid composition will be determined by the occurrence of adverse events, physical and neurological examinations (including EEG, neurocognitive tests, and SCAT5), clinical laboratory tests, vital sign measurements, and electrocardiograms (ECGs) (FIG. 4). Plasma amino acid levels and amino acid metabolite concentrations will be used to determine the pharmacokinetic properties of the composition. Changes in serum biomarkers (tau, NF-L, UCH-L1, and GFAP) associated with neural inflammation will also be assessed.

This 15 day study will utilize a single-blind, placebo controlled 2:1 (i.e. n=20 amino acid composition; n=10 placebo) randomization design to assess the efficacy, safety, and tolerability of the amino acid composition or placebo (excipient and color matched to the amino acid composition) administered orally 2 times a day (BID) in adult subjects with mTBI. For each dose, three (3) stick packs of the amino acid composition will be dissolved in approximately 12 ounces of water and taken one hour after breakfast and one hour after dinner. For enrollment, subjects will have a documented diagnosis of mTBI (e.g., a concussion) due to a sports-related and/or a blunt trauma to the head as determined by a trained health professional (e.g. ER/acute care facility/team physician/physiotherapist) using their local concussion protocol. The diagnosis of mTBI will be confirmed by a study investigator within 24 hrs or less of the injury event. On study Day 1, subjects may receive up to 3 administrations of study product orally without regard to meals (e.g., first administration within 30 minutes after randomization and ideally within 24 hours of the injury event; second administration within 1-3 hours of first administration but prior to discharge from ER/acute care facility/study site; and third administration is self-administered one hour after dinner or at bedtime). On Days 2-7, study product will continue to be self-administered orally 2 times a day (BID): one hour after breakfast and one hour after dinner. Subjects will return to the study site on Day 8 following an overnight fast of at least eight (8) hours for pharmacokinetic assessments.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A composition comprising:
a) a Branched Chain Amino Acid (BCAA) entity chosen from a leucine amino acid entity (chosen from L-leucine, oxo-leucine (alpha-ketoisocaproate (KIC)), HMB (beta-hydroxy-beta-methylbutyrate), oxo-leucine, isovaleryl-CoA, and N-acetyl-leucine or salts thereof), an isoleucine amino acid entity (chosen from L-isoleucine, 2-oxo-3-methyl-valerate (alpha-keto-beta-methylvaleric acid (KMV)), threonine, 2-oxo-3-methyl-valerate, methylbutyryl-CoA, and N-acetyl-isoleucine or salts thereof), a valine amino acid entity (chosen from L-valine, 2-oxo-valerate (alpha-ketoisovalerate (KIV)), isobutyryl-CoA, and N-acetyl-valine or salts thereof), or a combination of two or three BCAA entities;
b) a N-acetylcysteine (NAC) entity (chosen from N-acetylcysteine, serine, acetylserine, cystathionine, glutathione, homocysteine, methionine, L-cysteine, cystine, and cysteamine or salts thereof); and
c) acetyl-1-carnitine (ALCAR) or a salt thereof, or a dipeptide or salt thereof or a tripeptide or salt thereof comprising ALCAR;
wherein at least one amino acid entity of (a)-(c) is not provided as a peptide of more than 20 amino acid residues in length,
wherein the total wt. % of (a)-(c) is greater than the total wt. % of other protein components or non-protein components in the composition (in dry form), and
wherein one or two amino acid entities in (a)-(c) are in one or both of free amino acid form or salt amino acid form in the composition.

2. The composition of claim 1, further comprising (d) a creatine entity, optionally wherein the creatine entity is not provided as a peptide of more than 20 amino acid residues in length.

3. A composition comprising:
a) leucine, isoleucine, and valine;
b) N-acetylcysteine (NAC); and
c) acetyl-L-carnitine.

4-6. (canceled)

7. The composition of claim 1, wherein the composition does not comprise a peptide of more than 20 amino acid residues in length, or if a peptide of more than 20 amino acid residues in length is present the peptide is present at less than 10 wt. % or less of the total wt. of protein components or total components of the composition (in dry form).

8. The composition of claim 1, wherein the composition comprises a combination of 19 or fewer amino acid entities.

9. (canceled)

10. The composition of claim 1, wherein the wt. % of the NAC entity is at least 3 wt. % of the total wt. of protein components or total components in the composition (in dry form).

11. The composition of claim 1, wherein the wt. % of the ALCAR or a salt thereof or a dipeptide or salt thereof, or a tripeptide or salt thereof, comprising ALCAR is at least 1 wt. % of the total wt. of protein components or total components in the composition (in dry form).

12. The composition of claim 1, wherein the composition comprises all three BCAA entities, the NAC entity, and the ALCAR, and the wt. ratio of the leucine amino acid entity:isoleucine amino acid entity:the valine amino acid entity:NAC entity:ALCAR, is 10+/−15%:10+/−15%:10+/−15%:3+/−15%:1.2+/−15%, where the ratios are determined based on an equivalent amount of each amino acid in free form.

13-15. (canceled)

16. The composition of claim 1, wherein in the composition:
a) the leucine amino acid entity is chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) the NAC entity is NAC or a salt thereof or a dipeptide or salt thereof, or a tripeptide or salt thereof, comprising NAC; and
c) the ALCAR entity is ALCAR or a salt thereof or a dipeptide or salt thereof, or a tripeptide or salt thereof, comprising ALCAR.

17. The composition of claim 16, wherein in the composition:
d) the isoleucine amino acid entity, if present, is L-isoleucine or a salt thereof or a dipeptide or salt thereof, or tripeptide or salt thereof, comprising L-isoleucine; and
e) the valine amino acid entity, if present, is L-valine or a salt thereof or a dipeptide or salt thereof, or tripeptide or salt thereof, comprising L-valine.

18. The composition of claim 16, wherein the composition further comprises creatine or a salt thereof or a dipeptide or salt thereof, or a tripeptide or salt thereof, comprising creatine.

19. The composition of claim 1, wherein the composition comprises: L-leucine or a salt thereof, L-isoleucine or a salt thereof, L-valine or a salt thereof, NAC or a salt thereof, and ALCAR or a salt thereof.

20. The composition of claim 19, wherein the composition further comprises creatine or a salt thereof.

21. The composition of claim 1, wherein the composition is formulated with a pharmaceutically acceptable carrier.

22. A composition comprising:
a) a Branched Chain Amino Acid (BCAA) entity chosen from a leucine amino acid entity (chosen from L-leucine, oxo-leucine (alpha-ketoisocaproate (KIC)), HMB (beta-hydroxy-beta-methylbutyrate), oxo-leucine, isovaleryl-CoA, and N-acetyl-leucine or salts thereof), an isoleucine amino acid entity (chosen from L-isoleucine, 2-oxo-3-methyl-valerate (alpha-keto-beta-methylvaleric acid (KMV)), threonine, 2-oxo-3-methyl-valerate, methylbutyryl-CoA, and N-acetyl-isoleucine or salts thereof), a valine amino acid entity (chosen from L-valine, 2-oxo-valerate (alpha-ketoisovalerate (KIV)), isobutyryl-CoA, and N-acetyl-valine or salts thereof), or a combination of two or three BCAA entities;
b) a N-acetylcysteine (NAC) entity (chosen from N-acetylcysteine, serine, acetylserine, cystathionine, glutathione, homocysteine, methionine, L-cysteine, cystine, and cysteamine or salts thereof); and
c) acetyl-1-carnitine (ALCAR) or a salt thereof, or a dipeptide or salt thereof or a tripeptide or salt thereof comprising ALCAR;
wherein at least one amino acid entity of (a)-(c) is not provided as a peptide of more than 20 amino acid residues in length,
wherein the total wt. % of (a)-(c) is greater than the total wt. % of other protein components or non-protein components in the composition (in dry form),
wherein one or two amino acid entities in (a)-(c) are in one or both of free amino acid form or salt amino acid form in the composition, and
wherein the composition is formulated as a dietary composition.

23. A method of improving one or both of neuronal function or cognitive function, comprising administering to a subject an effective amount of a composition comprising:
a) a Branched Chain Amino Acid (BCAA) entity chosen from a leucine amino acid entity (chosen from L-leucine, oxo-leucine (alpha-ketoisocaproate (KIC)), HMB (beta-hydroxy-beta-methylbutyrate), oxo-leucine, isovaleryl-CoA, and N-acetyl-leucine or salts thereof), an isoleucine amino acid entity (chosen from L-isoleucine, 2-oxo-3-methyl-valerate (alpha-keto-beta-methylvaleric acid (KMV)), threonine, 2-oxo-3-methyl-valerate, methylbutyryl-CoA, and N-acetyl-isoleucine or salts thereof), a valine amino acid entity (chosen from L-valine, 2-oxo-valerate (alpha-ketoisovalerate (KIV)), isobutyryl-CoA, and N-acetyl-valine or salts thereof), or a combination of two or three BCAA entities;
b) a N-acetylcysteine (NAC) entity (chosen from N-acetylcysteine, serine, acetylserine, cystathionine, glutathione, homocysteine, methionine, L-cysteine, cystine, and cysteamine or salts thereof); and
c) an acetyl-1-carnitine (ALCAR) entity (chosen from acetyl-L-carnitine, lysine, trimethyllysine, trimethyl-3-OH-lysine, carnitine, O-acyl-carnitine, acyl-CoA, citrate, succinate, C3-carnitine, C5-carnitine, C4-dicarboxylcarnitine, C6-carnitine, C8-carnitine, C12:1 acylcarnitine, C14 acylcarnitine, C14:1 acylcarnitine, C16 acylcarnitine, C18:2 acylcarnitine, C18:1 acylcarnitine, and C18 acylcarnitine);
wherein at least one amino acid entity of (a)-(c) is not provided as a peptide of more than 20 amino acid residues in length,
wherein the total wt. % of (a)-(c) is greater than the total wt. % of other protein components or non-protein components in the composition (in dry form), and
wherein one, two, or three amino acid entities in (a)-(c) are in one or both of free amino acid form or salt amino acid form in the composition,
thereby improving one or both of neuronal function or cognitive function in the subject.

24. The method of claim 23, wherein the subject has a symptom of traumatic brain injury (TBI) selected from the group consisting of deficits, dizziness, hearing loss, headache (e.g., frequent headache), loss of consciousness, memory loss, confusion, sleep disturbance, nausea, decreased balance, fatigue, drowsiness, blurred vision, ringing in ears, sensitivity to light, sensitivity to sound, decreased ability to concentration, mood swings, and increased anxiety,
wherein the method improves the symptom.

25. The method of claim 23, wherein the subject has a symptom of stroke selected from the group consisting of numbness, decreased balance, memory loss, facial weakness, eyelid dropping, paralysis (e.g., hemiplegia), decreased sensory sensation, decreased reflexes, tongue weakness, involuntary eye moment, a visual field defect, aphasia, increased confusion, vertigo, decreased speaking ability (e.g., apraxia), decreased walking ability, and decreased motor coordination,
wherein the method improves the symptom.

26. A method for treating or preventing neuronal injury, comprising administering to a subject in need thereof an effective amount of a composition comprising:
a) a Branched Chain Amino Acid (BCAA) entity chosen from a leucine amino acid entity (chosen from L-leucine, oxo-leucine (alpha-ketoisocaproate (KIC)), HMB (beta-hydroxy-beta-methylbutyrate), oxo-leucine, isovaleryl-CoA, and N-acetyl-leucine or salts thereof), an isoleucine amino acid entity (chosen from L-isoleucine, 2-oxo-3-methyl-valerate (alpha-keto-beta-methylvaleric acid (KMV)), threonine, 2-oxo-3-methyl-valerate, methylbutyryl-CoA, and N-acetyl-isoleucine or salts thereof), a valine amino acid entity (chosen from L-valine, 2-oxo-valerate (alpha-ketoisovalerate (KIV)), isobutyryl-CoA, and N-acetyl-valine or salts thereof), or a combination of two or three BCAA entities;
b) a N-acetylcysteine (NAC) entity (chosen from N-acetylcysteine, serine, acetylserine, cystathionine, glutathione, homocysteine, methionine, L-cysteine, cystine, and cysteamine or salts thereof); and
c) an acetyl-1-carnitine (ALCAR) entity (chosen from acetyl-L-carnitine, lysine, trimethyllysine, trimethyl-3-OH-lysine, carnitine, O-acyl-carnitine, acyl-CoA, citrate, succinate, C3-carnitine, C5-carnitine, C4-dicarboxylcarnitine, C6-carnitine, C8-carnitine, C12:1 acylcarnitine, C14 acylcarnitine, C14:1 acylcarnitine, C16 acylcarnitine, C18:2 acylcarnitine, C18:1 acylcarnitine, and C18 acylcarnitine);
wherein at least one amino acid entity of (a)-(c) is not provided as a peptide of more than 20 amino acid residues in length,
wherein the total wt. % of (a)-(c) is greater than the total wt. % of other protein components or non-protein components in the composition (in dry form), and
wherein one, two, or three amino acid entities in (a)-(c) are in one or both of free amino acid form or salt amino acid form in the composition,
thereby treating or preventing the neuronal injury.

27. The method of claim 23, wherein the subject has been diagnosed as having TBI.

28. The method of claim 27, wherein the TBI is mild TBI.

29. (canceled)

30. The method of claim 23, wherein the subject is at risk of or has stroke, and if the subject has stroke wherein the stroke is ischemic stroke, acute ischemic stroke, or transient ischemic attack.

31-36. (canceled)
